(19) United States
(12) Patent Application Publication
(10) Pub. No.: US 2024/0174672 A1
(43) Pub. Date: May 30, 2024

(54) Spirocycle Containing Pyridone Compounds

(71) Applicant: Protego Biopharma, Inc., San Diego, CA (US)

(72) Inventors: Huang QIU, San Diego, CA (US); Steven J. Wilkens, San Diego, CA (US); Hank Michael James Petrassi, La Jolla, CA (US); Richard F. Labaudiniere, Charleston, SC (US); Bo Qin, La Jolla, CA (US)

(21) Appl. No.: 18/494,587

(22) Filed: Oct. 25, 2023

Related U.S. Application Data
(60) Provisional application No. 63/381,010, filed on Oct. 26, 2022.

(51) Int. Cl. C07D 471/10; A61K 45/06; C07D 487/10; C07D 519/00

(52) U.S. Cl. CPC C07D 471/10; A61K 45/06; C07D 487/10; C07D 519/00

(57) ABSTRACT

Provided herein are compounds that stabilize immunoglobulin light chains, and pharmaceutically acceptable derivatives thereof. Also provided are pharmaceutical compositions containing the compounds and methods of using the compounds for treating a subject with light chain amyloidosis.

### RELATED APPLICATION

This application claims benefit of priority to U.S. provisional patent application No. 63/381,010, filed Oct. 26, 2022. The contents of the above-referenced application are incorporated by reference herein in their entirety.

### FIELD

Provided herein are compounds useful for treatment of light chain amyloidosis. In one embodiment, the compounds are spirocycle containing pyridone compounds.

### BACKGROUND

Light chain (LC) amyloidosis (AL amyloidosis) is a progressive and often fatal degenerative disease caused by monoclonal plasma cell proliferation, resulting in an abnormal free light chain (FLC) ratio and conformational changes within involved immunoglobulin light chains (iFLC) after secretion by clonal plasma cells that result in organ toxicity, e.g., cardiomyopathy, nephrotic syndrome and end-stage renal failure. Organ damage remains the major source of mortality and morbidity. Lambda light chains are more often amyloidogenic than the kappa light chains (˜80%). The light chain conformational changes also often lead to light chain aggregation, which may also drive proteotoxicity in some post-mitotic tissues. The pathologic mechanisms of disease leading to organ toxicity include both toxicity of amyloidogenic LC and mass effects of deposits, both modulated by misfolded LC concentration.

Light chain amyloidosis patients are treated today by targeting the cancer component of this disease (proliferating clonal plasma cells) employing chemotherapy cocktails typically involving proteasome inhibitors (and, when possible, stem cell transplants), which ideally eliminate the clonal plasma cells secreting full-length light chains. However, complete clonal plasma cell eradication is achieved in only 30-40% of the patients and most eventually relapse. Restoration of organ function in treated patients is highly variable and often incomplete, resulting in poor outcomes. The current hematologic response criteria for AL amyloidosis define complete response (CR) as no evidence of monoclonal protein based on serum and urine immunofixation, as well as achieving a normal FLC ratio. The response criteria do not take the levels of iFLC into consideration. It has been shown that increased levels of iFLCs at the time of normal FLC ratio and complete or very good partial hematological response are associated with inferior incomes, i.e., lower organ response and lower overall survival. However, even low levels of amyloidogenic monoclonal FLC can result in organ dysfunction. Moreover, light chain amyloidosis patients exhibiting cardiac involvement are often too sick to tolerate chemotherapy and die within a year of diagnosis.

Thus, there is a need for additional treatments of light chain amyloidosis.

### SUMMARY

Provided herein are compounds for use in compositions and methods of treatment of light chain amyloidosis (AL amyloidosis). In certain embodiments, the compounds provided herein are light chain kinetic stabilizers. The compounds provided herein are spirocycle containing pyridone compounds. In one embodiment, the compounds for use in the compositions and methods provided herein have formula I:

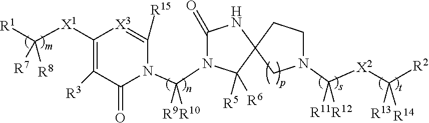

or a pharmaceutically acceptable derivative thereof, where R¹ to R¹⁵, X¹, X², X³, m, n, p, s and t are as defined herein.

In another embodiment, the compounds for use in the compositions and methods provided herein have formula II:

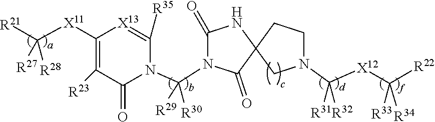

or a pharmaceutically acceptable derivative thereof, where R²¹ to R²⁴, R²⁷ to R³⁵, X¹¹, X¹², X¹³, a, b, c, d and f are as defined herein.

In one embodiment, provided herein is a method of treating light chain amyloidosis by administering to a subject a compound or composition provided herein. In another embodiment, provided herein is a method of stabilizing immunoglobulin light chains by contacting the immunoglobulin light chains with a compound provided herein. In one embodiment, the immunoglobulin light chains are stabilized in a native conformation thereof. In certain embodiments, the immunoglobulin light chains are dimers. In another embodiment, provided herein is a method of preventing or lessening immunoglobulin light chain misfolding and/or endoproteolysis by contacting the immunoglobulin light chains with a compound provided herein. In another embodiment, provided is a method of maintenance therapy upon recurrence of light chain amyloidosis following primary treatment by administering to a subject a compound or composition provided herein. In another embodiment, provided is a method of combination therapy using a compound or composition provided herein in combination with one or more additional active agents that treat light chain amyloidosis, deplete clonal plasma cells, stabilize immunoglobulin light chains, prevent or lessen immunoglobulin light chain misfolding and/or endoproteolysis, promote clearance of fibrils, or that are effective in maintenance therapy upon recurrence of light chain amyloidosis following primary treatment.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating light chain amyloidosis.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

As used herein, the EC₅₀ refers to an amount, concentration or dosage of a particular test compound that achieves 50% of a maximal response in an assay that measures such response. Transient misfolding events in the unstable WIL-FL LC₂ enables proteinase K proteolysis, resulting in release of smaller fluorescein-labeled peptides that exhibit decreased fluorescence polarization. Small molecules that bind to and kinetically stabilize WIL-FL LC₂ decrease the rate at which the LC is proteolyzed, and therefore maintain the high fluorescence polarization signal longer.

As used herein, fold protection refers to the calculation as described in Example 1 Proteinase K Sensitivity Assay.

As used herein, the IC₅₀ refers to an amount, concentration or dosage of a particular test compound that achieves a 50% inhibition of a maximal response in an assay that measures such response.

As used herein, the K_{d} refers to the measured equilibrium dissociation constant between a compound (or ligand) and a protein (or binding domain of a protein).

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbomanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently.

The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R′″ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″—, wherein Q′ and Q″ are independently —NR—, —O—, —CRR′— or a single bond, and q is an integer of from 0 to 3.

Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ᵤ—X′—(CR″R′″)ᵥ—, where u and v are independently integers from 0 to 3, and X′ is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

### II. Compounds for Use in Compositions and Methods

In one embodiment, provided herein is a compound for use in the compositions and methods provided herein having Formula I:

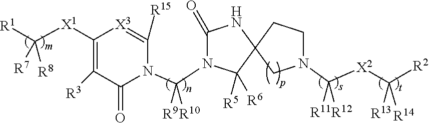

or a pharmaceutically acceptable derivative thereof, wherein
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl;
R³ to R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, s and t are each 0, X² is a bond and the compound for use in the compositions and methods provided herein has Formula Ia:

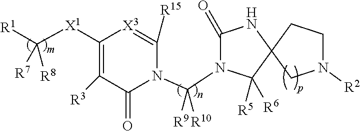

or a pharmaceutically acceptable derivative thereof, wherein
n is an integer from 1-4;
p is an integer from 1-3;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

In another embodiment, p is 2, s and t are each 0, X² is a bond and the compound for use in the compositions and methods provided herein has Formula Ib:

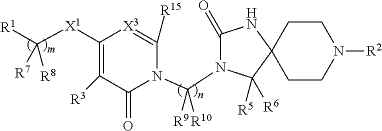

or a pharmaceutically acceptable derivative thereof, wherein
n is an integer from 1-4;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

In another embodiment, each of R³ to R⁶, R⁹ and R¹⁰ are H, p is 2, s and t are each 0, X² is a bond and the compound for use in the compositions and methods provided herein has Formula Ic:

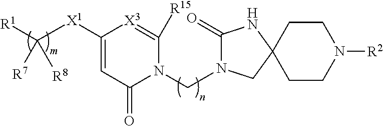

or a pharmaceutically acceptable derivative thereof, wherein
n is an integer from 1-4;
m is an integer from 0-3;
X¹ is a bond, O, NR¹⁶ or CO;
X³ is N or CH;
R¹ is aryl, heteroaryl or heterocycloalkyl;
R² is heteroaryl;
R⁷ and R⁸ are each independently H, alkyl or aralkyl, or together with the carbon atom to which they are attached form cycloalkyl; and
R¹⁵ and R¹⁶ are each independently H or alkyl.

In another embodiment, R¹ in Formulae I, Ia, Ib or Ic is aryl or heterocycloalkyl. In another embodiment, R¹ in Formulae I, Ia, Ib or Ic is aryl. In another embodiment, R¹ in Formulae I, Ia, Ib or Ic is heterocycloalkyl.

In another embodiment, X¹ is a bond, each of R³ to R⁶, R⁹ and R¹⁰ are H, p is 2, m, s and t are each 0, X² is a bond and the compound for use in the compositions and methods provided herein has Formula Id:

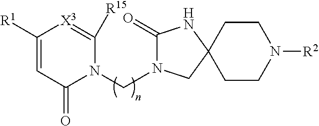

or a pharmaceutically acceptable derivative thereof, wherein
n is 2 or 3;
X³ is N or CH;
R¹ is heterocycloalkyl, optionally substituted with 1-3 substituents each independently selected from alkyl and aryl;
R² is heteroaryl; and
R¹⁵ is alkyl.

In another embodiment, the compound for use in the compositions and methods provided herein has Formula Ie:

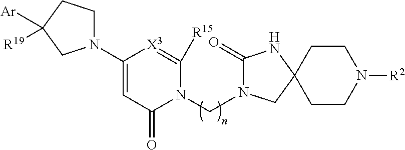

or a pharmaceutically acceptable derivative thereof, wherein
n is 2 or 3;
X³ is N or CH;
Ar is aryl;
R² is heteroaryl;
R¹⁵ is alkyl; and
R¹⁹ is alkyl.

In another embodiment, R¹⁹ is lower alkyl. In another embodiment, R¹⁹ is methyl or ethyl. In another embodiment, R¹⁹ is methyl. In another embodiment, R¹⁹ is ethyl.

In another embodiment, the compound for use in the compositions and methods provided herein has the formula:

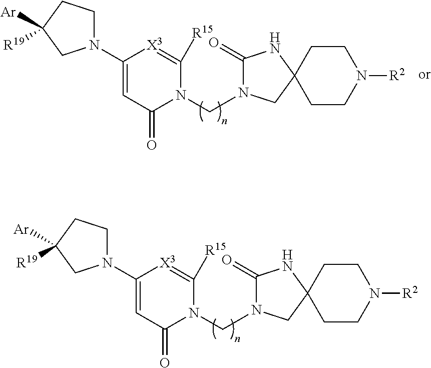

where the variables are as defined herein.

In another embodiment, the compound for use in the compositions and methods provided herein has Formula If:

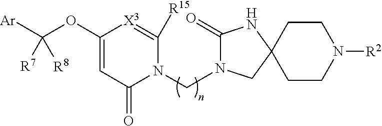

or a pharmaceutically acceptable derivative thereof, wherein
n is 2 or 3;
X³ is N or CH;
Ar is aryl;
R² is heteroaryl;
R¹⁵ is alkyl; and
R⁷ and R⁸ are each independently H or alkyl, or together with the carbon atom to which they are attached form cycloalkyl.

In another embodiment, R⁷ is alkyl and R⁸ is H. In another embodiment, R⁷ is methyl and R⁸ is H. In another embodiment, R⁷ and R⁸ together with the carbon atom to which they are attached form cyclopropyl.

In another embodiment, the compound for use in the compositions and methods provided herein has the formula:

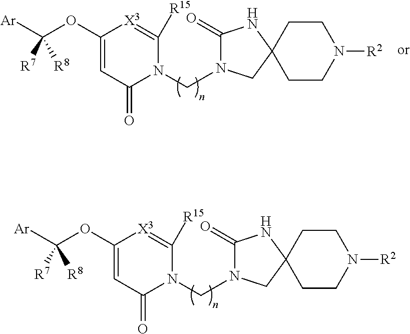

where the variables are as defined herein.

In another embodiment, p is 1, s and t are each 0, X² is a bond and the compound for use in the compositions and methods provided herein has Formula Ig:

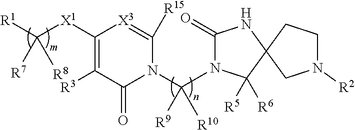

or a pharmaceutically acceptable derivative thereof, wherein
n is an integer from 1-4;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

In another embodiment, each of R³ to R⁶, R⁹ and R¹⁰ are H, p is 1, s and t are each 0, X² is a bond and the compound for use in the compositions and methods provided herein has Formula Ih:

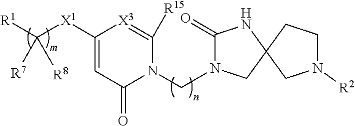

or a pharmaceutically acceptable derivative thereof, wherein
n is an integer from 1-4;
m is an integer from 0-3;
X¹ is a bond, O, NR¹⁶ or CO;
X³ is N or CH;
R¹ is aryl, heteroaryl or heterocycloalkyl;
R² is heteroaryl;
R⁷ and R⁸ are each independently H, alkyl or aralkyl, or together with the carbon atom to which they are attached form cycloalkyl; and
R¹⁵ and R¹⁶ are each independently H or alkyl.

In another embodiment, R¹ in Formulae Ig or Ih is aryl or heterocycloalkyl. In another embodiment, R¹ in Formulae Ig or Ih is aryl. In another embodiment, R¹ in Formulae Ig or Ih is heterocycloalkyl.

In another embodiment, X¹ is a bond, each of R³ to R⁶, R⁹ and R¹⁰ are H, p is 1, m, s and t are each 0, X² is a bond and the compound for use in the compositions and methods provided herein has Formula Ii:

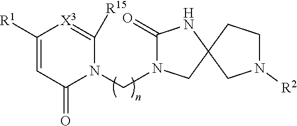

or a pharmaceutically acceptable derivative thereof, wherein
n is 2 or 3;
X³ is N or CH;
R¹ is heterocycloalkyl, optionally substituted with 1-3 substituents each independently selected from alkyl and aryl;
R² is heteroaryl; and
R¹⁵ is alkyl.

In another embodiment, the compound for use in the compositions and methods provided herein has Formula Ij:

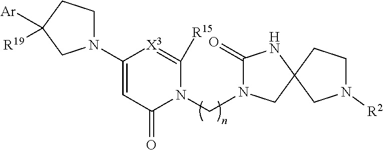

or a pharmaceutically acceptable derivative thereof, wherein
n is 2 or 3;
X³ is N or CH;
Ar is aryl;
R² is heteroaryl;
R¹⁵ is alkyl; and
R¹⁹ is alkyl.

In another embodiment, R¹⁹ is lower alkyl. In another embodiment, R¹⁹ is methyl or ethyl. In another embodiment, R¹⁹ is methyl. In another embodiment, R¹⁹ is ethyl.

In another embodiment, the compound for use in the compositions and methods provided herein has the formula:

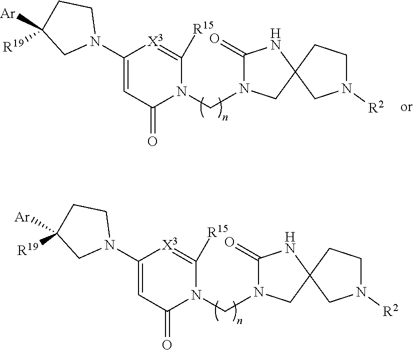

where the variables are as defined herein.

In another embodiment, the compound for use in the compositions and methods provided herein has Formula Ik:

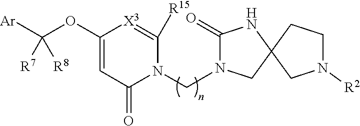

or a pharmaceutically acceptable derivative thereof, wherein
n is 2 or 3;
X³ is N or CH;
Ar is aryl;
R² is heteroaryl;
R¹⁵ is alkyl; and
R⁷ and R⁸ are each independently H or alkyl, or together with the carbon atom to which they are attached form cycloalkyl.

In another embodiment, R⁷ is alkyl and R⁸ is H. In another embodiment, R⁷ is methyl and R⁸ is H. In another embodiment, R⁷ and R⁸ together with the carbon atom to which they are attached form cyclopropyl.

In another embodiment, the compound for use in the compositions and methods provided herein has the formula:

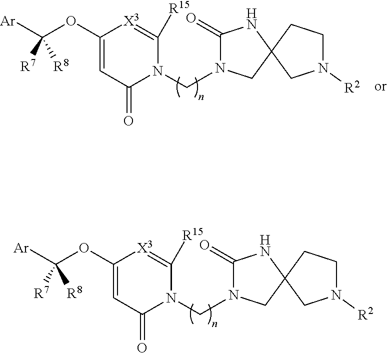

where the variables are as defined herein.

In another embodiment, n is 2. In another embodiment, n is 3.

In another embodiment, X³ is N. In another embodiment, X³ is CH.

In another embodiment, Ar is unsubstituted aryl. In another embodiment, Ar is optionally substituted phenyl. In another embodiment, Ar is unsubstituted phenyl.

In another embodiment, R² is bicyclic or monocyclic heteroaryl. In another embodiment, R² is triazolopyrazinyl, imidazopyrazinyl, pyrazolopyrimidinyl, pyridyl, pyridazinyl or pyrimidinyl. In another embodiment, R² is triazolopyrazinyl, imidazopyrazinyl, pyrazolopyrimidinyl or pyrimidinyl. In another embodiment, R² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl or 2-pyrimidinyl. In another embodiment, R² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl, 2-pyridyl, 3-pyridyl, 3-pyridazinly or 2-pyrimidinyl. In another embodiment, R² is [1,2,4]-triazolo[4,3-a]pyrazin-8-yl, imidazo[1,5-a]pyrazin-8-yl, 5-carboxyimidazo[1,5-a]pyrazin-8-yl, imidazo[1,2-a]pyrazin-8-yl, 1H-pyrazolo[3,4-d]pyrimidin-4-yl, 6-carboxy-3-pyridyl, 6-carboxy-2-pyridyl, 6-ethoxycarbonyl-2-pyridyl, 6-ethoxycarbonyl-3-pyridyl, 6-carboxy-3-pyridazinyl, 6-ethoxycarbonyl-3-pyridazinyl or 2-pyrimidinyl.

In another embodiment, R¹⁵ is lower alkyl. In another embodiment, R¹⁵ is methyl.

In one embodiment, provided herein is a compound for use in the compositions and methods provided herein having Formula Im:

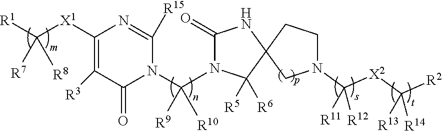

or a pharmaceutically acceptable derivative thereof, wherein
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl; and
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula Im, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula Im, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁸ to R¹⁴ are each independently H; and
R⁷ and R¹⁵ are each independently alkyl.

In another embodiment, the compounds provided herein for use in the compositions and methods provided herein have Formula Im, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula Im, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁸ to R¹⁴ are each independently H; and
R⁷ and R¹⁵ are each independently alkyl.

In another embodiment, provided herein is a compound for use in the compositions and methods provided herein having Formula In:

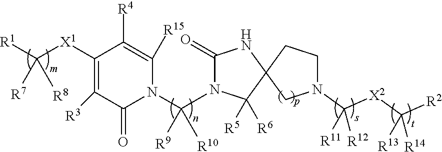

or a pharmaceutically acceptable derivative thereof, wherein
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl;
R³ to R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula In, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶ and R⁹ to R¹⁵ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula In, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl; and
R³ to R⁵, R⁶ and R⁸ to R¹⁴ are each independently H; and
R⁷ and R¹⁵ are each independently alkyl.

In another embodiment, the compounds provided herein for use in the compositions and methods provided herein have Formula In, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶ and R⁹ to R¹⁵ are each independently H, alkyl or aralkyl; and
R⁷ and R⁸ are each independently H, alkyl or aralkyl, or together with the carbon atom to which they are attached form cycloalkyl.

In another embodiment, the compounds provided herein for use in the compositions and methods provided herein have Formula In, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl; and
R³ to R⁵, R⁶ and R⁸ to R¹⁴ are each independently H; and
R⁷ and R¹⁵ are each independently alkyl.

In another embodiment, s and t are each 0, X² is a bond, X³ is CR⁴ and the compounds for use in the compositions and methods provided herein have Formula Io:

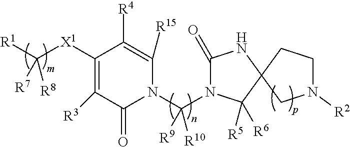

wherein:
n is an integer from 1-4;
p is an integer from 1-3;
m is an integer from 0-3;
X¹ is a bond, O, NR¹⁶ or CO;
R¹ is aryl or heterocycloalkyl;
R² is heteroaryl; and
R³ to R¹⁰, R¹⁵ and R¹⁶ are each independently H or alkyl.

In another embodiment, s and t are each 0, X² is a bond, X³ is N and the compounds for use in the compositions and methods provided herein have Formula Ip:

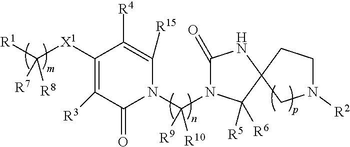

wherein:
n is an integer from 1-4;
p is an integer from 1-3;
m is an integer from 0-3;
X¹ is a bond, O, NR¹⁶ or CO;
R¹ is aryl or heterocycloalkyl;
R² is heteroaryl; and
R³, R⁵ to R¹⁰, R¹⁵ and R¹⁶ are each independently H or alkyl.

In another embodiment, the compounds for use in the compositions and methods provided herein have Formula Io or Ip, wherein:
n is 2 or 3;
p is 2;
m is 0 or 1;
X¹ is a bond or O;
R¹ is aryl or heterocycloalkyl, each optionally substituted with 1-3 substituents each independently selected from alkyl and aryl;
R² is heteroaryl; and
R³, R⁵ to R¹⁰, R¹⁵ and R¹⁶ are each independently H or alkyl.

In another embodiment, the compounds for use in the compositions and methods provided herein have Formula Io or Ip, wherein:
n is 2 or 3;
p is 1;
m is 0 or 1;
X¹ is a bond or O;
R¹ is aryl or heterocycloalkyl, each optionally substituted with 1-3 substituents each independently selected from alkyl and aryl;
R² is heteroaryl; and
R³, R⁵ to R¹⁰, R¹⁵ and R¹⁶ are each independently H or alkyl.

In one embodiment, the compounds provided herein for use in the compositions and methods provided herein those shown in Table 1 below:

| Compound | Structure |
|---|---|
| 1 | 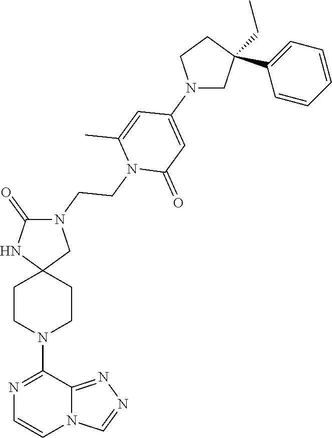 [SMILES: CC[C@@]1(c2ccccc2)CCN(c2cc(C)n(CCN3CC4(CCN(c5nccn6cnnc56)CC4)NC3=O)c(=O)c2)C1] |
| 2 | 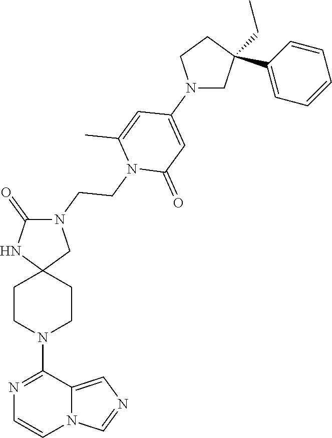 [SMILES: CC[C@@]1(c2ccccc2)CCN(c2cc(C)n(CCN3CC4(CCN(c5nccn6cncc56)CC4)NC3=O)c(=O)c2)C1] |
| 3 | 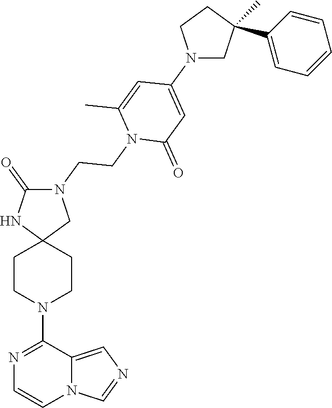 [SMILES: Cc1cc(N2CC[C@@](C)(c3ccccc3)C2)cc(=O)n1CCN1CC2(CCN(c3nccn4cncc34)CC2)NC1=O] |
| 4 | 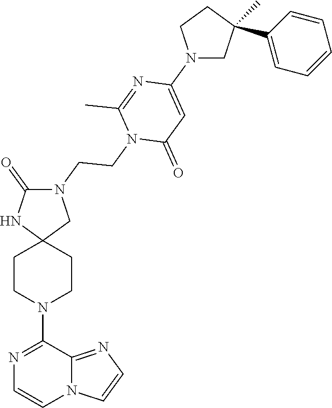 [SMILES: Cc1nc(N2CC[C@@](C)(c3ccccc3)C2)cc(=O)n1CCN1CC2(CCN(c3nccn4ccnc34)CC2)NC1=O] |
| 5 | 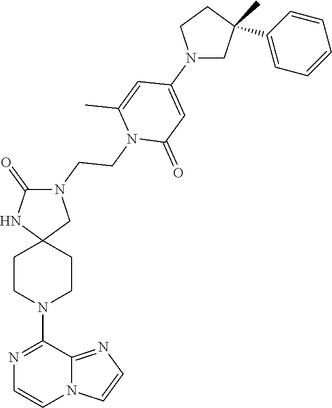 [SMILES: Cc1cc(N2CC[C@](C)(c3ccccc3)C2)cc(=O)n1CCN1CC2(CCN(c3nccn4ccnc34)CC2)NC1=O] |
| 6 | 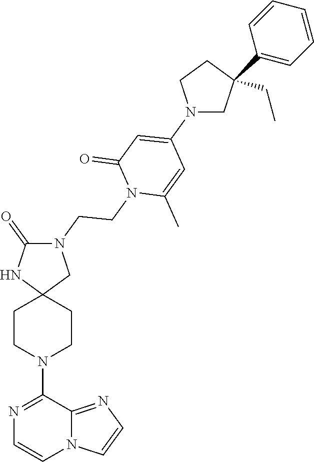 [SMILES: CC[C@@]1(c2ccccc2)CCN(c2cc(C)n(CCN3CC4(CCN(c5nccn6ccnc56)CC4)NC3=O)c(=O)c2)C1] |
| 7 | 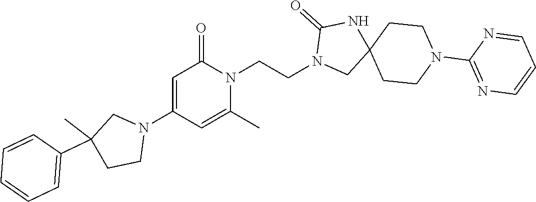 [SMILES: Cc1cc(N2CCC(C)(c3ccccc3)C2)cc(=O)n1CCN1CC2(CCN(c3ncccn3)CC2)NC1=O] |
| 8 | 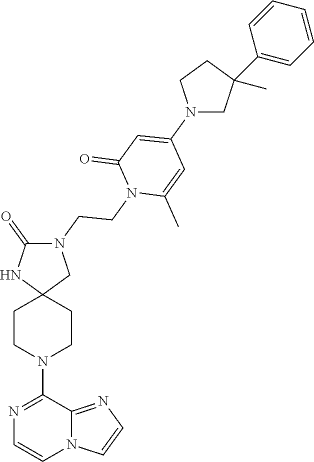 [SMILES: Cc1cc(N2CCC(C)(c3ccccc3)C2)cc(=O)n1CCN1CC2(CCN(c3nccn4ccnc34)CC2)NC1=O] |
| 9 | 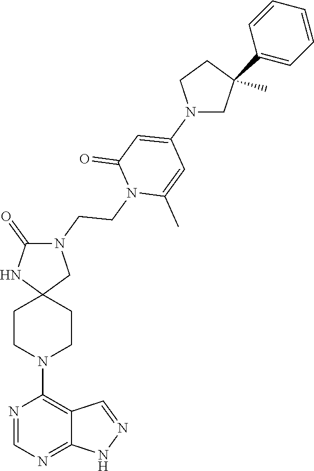 [SMILES: Cc1cc(N2CC[C@@](C)(c3ccccc3)C2)cc(=O)n1CCN1CC2(CCN(c3ncnc4[nH]ncc34)CC2)NC1=O] |
| 10 | 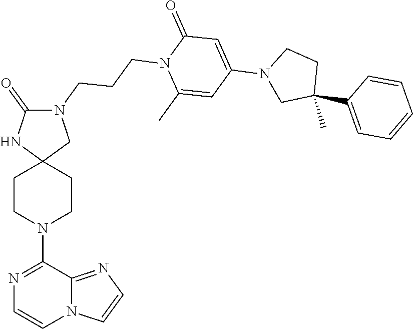 [SMILES: Cc1cc(N2CC[C@@](C)(c3ccccc3)C2)cc(=O)n1CCCN1CC2(CCN(c3nccn4ccnc34)CC2)NC1=O] |
| 11 | 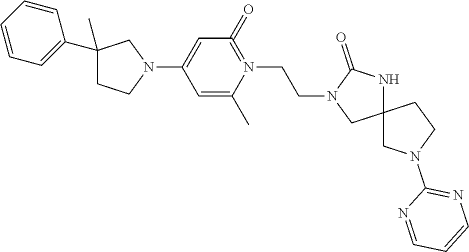 |
| 12 | 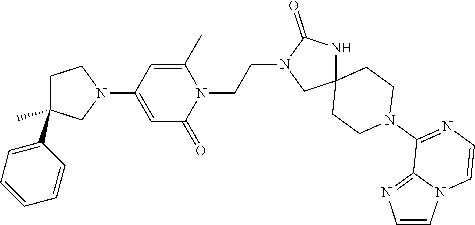 [SMILES: Cc1cc(N2CC[C@](C)(c3ccccc3)C2)cc(=O)n1CCN1CC2(CCN(c3nccn4ccnc34)CC2)NC1=O] |
| 13 | 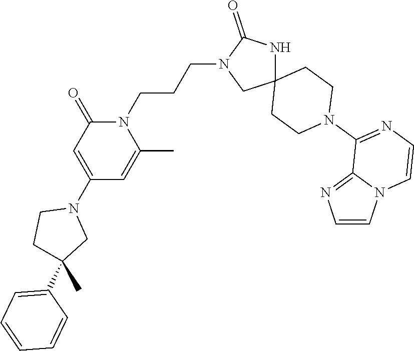 [SMILES: Cc1cc(N2CC[C@@](C)(c3ccccc3)C2)cc(=O)n1CCCN1CC2(CCN(c3nccn4ccnc34)CC2)NC1=O] |
| 14 | 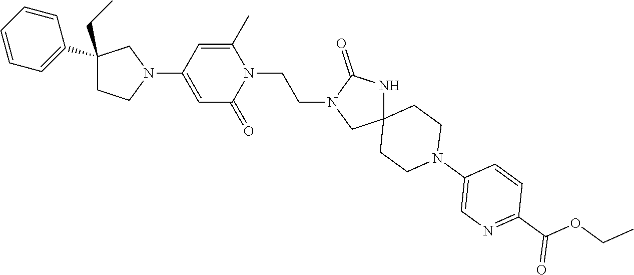 [SMILES: CCOC(=O)c1ccc(N2CCC3(CC2)CN(CCn2c(C)cc(N4CC[C@](CC)(c5ccccc5)C4)cc2=O)C(=O)N3)cn1] |
| 15 | 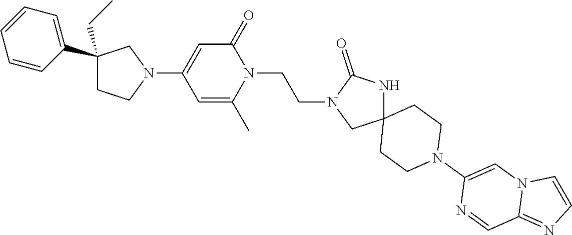 [SMILES: CC[C@@]1(c2ccccc2)CCN(c2cc(C)n(CCN3CC4(CCN(c5cn6ccnc6cn5)CC4)NC3=O)c(=O)c2)C1] |
| 16 | 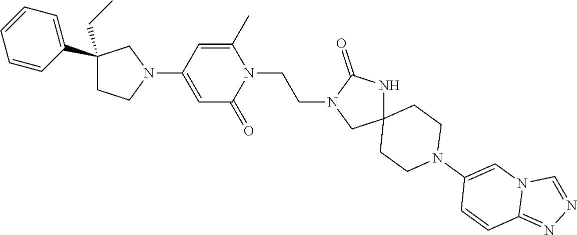 [SMILES: CC[C@@]1(c2ccccc2)CCN(c2cc(C)n(CCN3CC4(CCN(c5ccc6nncn6c5)CC4)NC3=O)c(=O)c2)C1] |
| 17 | 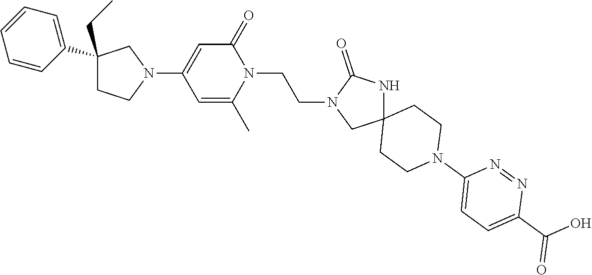 [SMILES: CC[C@]1(c2ccccc2)CCN(c2cc(C)n(CCN3CC4(CCN(c5ccc(C(=O)O)nn5)CC4)NC3=O)c(=O)c2)C1] |
| 18 | 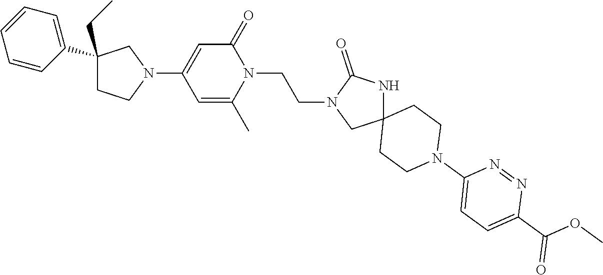 [SMILES: CC[C@]1(c2ccccc2)CCN(c2cc(C)n(CCN3CC4(CCN(c5ccc(C(=O)OC)nn5)CC4)NC3=O)c(=O)c2)C1] |
| 19 | 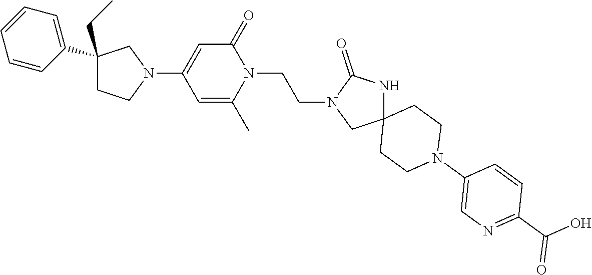 [SMILES: CC[C@]1(c2ccccc2)CCN(c2cc(C)n(CCN3CC4(CCN(c5ccc(C(=O)O)nc5)CC4)NC3=O)c(=O)c2)C1] |
| 20 | 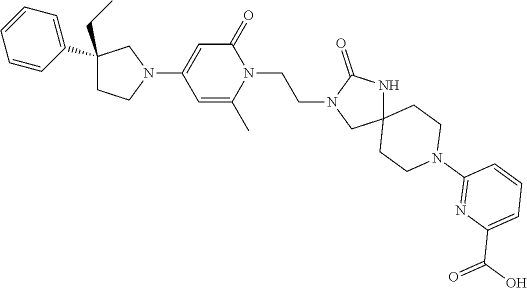 [SMILES: CC[C@]1(c2ccccc2)CCN(c2cc(C)n(CCN3CC4(CCN(c5cccc(C(=O)O)n5)CC4)NC3=O)c(=O)c2)C1] |
| 21 | 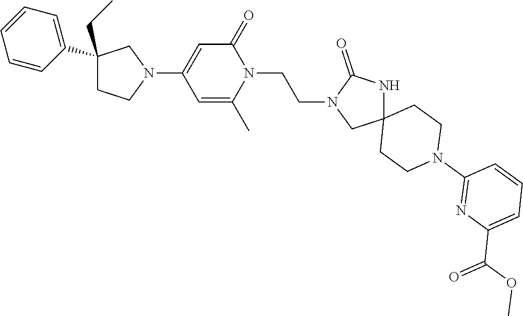 [SMILES: CC[C@]1(c2ccccc2)CCN(c2cc(C)n(CCN3CC4(CCN(c5cccc(C(=O)OC)n5)CC4)NC3=O)c(=O)c2)C1] |
| 22 | 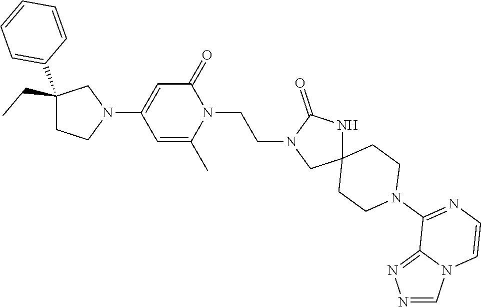 [SMILES: CC[C@]1(c2ccccc2)CCN(c2cc(C)n(CCN3CC4(CCN(c5nccn6cnnc56)CC4)NC3=O)c(=O)c2)C1] |
| 23 | 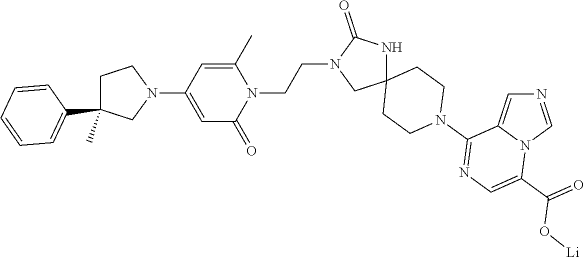 |
| 24 | 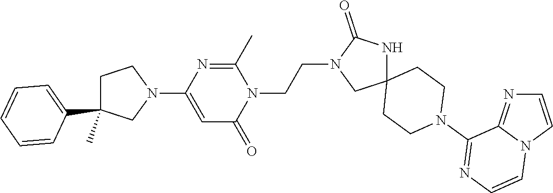 [SMILES: Cc1nc(N2CC[C@](C)(c3ccccc3)C2)cc(=O)n1CCN1CC2(CCN(c3nccn4ccnc34)CC2)NC1=O] |

In another embodiment, provided herein is a compound for use in the compositions and methods provided herein having Formula II:

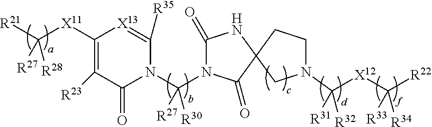

or a pharmaceutically acceptable derivative thereof, wherein:
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
X¹³ is N or CR²⁴;
R²¹ is aryl, heteroaryl or heterocycloalkyl;
R²² is aryl or heteroaryl; and
R²³ to R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

In another embodiment, d and f are each 0, X² is a bond and the compound for use in the compositions and methods provided herein has Formula IIa:

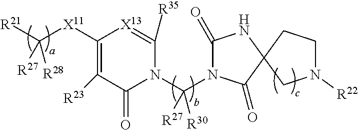

or a pharmaceutically acceptable derivative thereof, wherein:
b is an integer from 1-4;
c is an integer from 1-3;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl;
R²³ to R²⁵, R²⁹, R³⁰ and R³⁵ are each independently H, alkyl or aralkyl; and
R²⁷ and R²¹ are each independently H, alkyl or aralkyl, or together with the carbon atom to which they are attached form cycloalkyl.

In another embodiment, c is 2, d and f are each 0, X¹² is a bond and the compound for use in the compositions and methods provided herein has Formula IIb:

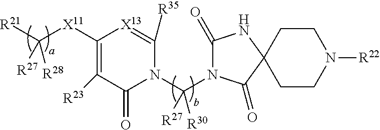

or a pharmaceutically acceptable derivative thereof, wherein:
b is an integer from 1-4;
a is an integer from 0-3;
X¹¹ is a bond, O, NR²⁵ or CO;
X¹³ is N or CR²⁴
R²¹ is aryl, heteroaryl or heterocycloalkyl;
R²² is heteroaryl; and
R²³ to R²⁵, R²⁹, R³⁰ and R³⁵ are each independently H, alkyl or aralkyl; and
R²⁷ and R²¹ are each independently H, alkyl or aralkyl, or together with the carbon atom to which they are attached form cycloalkyl.

In another embodiment, each of R²³, R²⁹ and R³⁰ are H, c is 2, d and f are each 0, X¹² is a bond and the compound for use in the compositions and methods provided herein has Formula IIc:

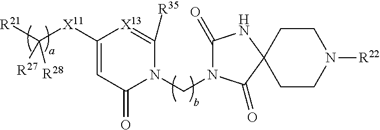

or a pharmaceutically acceptable derivative thereof, wherein:
b is an integer from 1-4;
a is an integer from 0-3;
X¹¹ is a bond, O, NR²⁵ or CO;
X¹³ is N or CH;
R²¹ is aryl, heteroaryl or heterocycloalkyl;
R²² is heteroaryl; and
R²⁵ and R³⁵ are each independently H or alkyl; and
R²⁷ and R²¹ are each independently H, alkyl or aralkyl, or together with the carbon atom to which they are attached form cycloalkyl.

In another embodiment, R²¹ in Formulae II, IIa, IIb or IIc is aryl or heterocycloalkyl. In another embodiment, R²¹ in Formulae II, IIa, IIb or IIc is aryl. In another embodiment, R²¹ in Formulae II, IIa, IIb or IIc is heterocycloalkyl.

In another embodiment, X¹¹ is a bond, each of R²³, R²⁹ and R³⁰ are H, c is 2, a, d and f are each 0, X¹² is a bond and the compound for use in the compositions and methods provided herein has Formula IId:

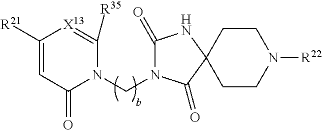

or a pharmaceutically acceptable derivative thereof, wherein:
b is 2 or 3;
X¹³ is N or CH;
R²¹ is heterocycloalkyl, optionally substituted with 1-3 substituents each independently selected from alkyl and aryl;
R²² is heteroaryl; and
R³⁵ is alkyl.

In another embodiment, the compound for use in the compositions and methods provided herein has Formula IIe:

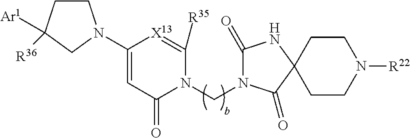

or a pharmaceutically acceptable derivative thereof, wherein:
b is 2 or 3;
X¹³ is N or CH;
Ar¹ is aryl;
R²² is heteroaryl;
R³⁵ is alkyl; and
R³⁶ is alkyl.

In another embodiment, R³⁶ is lower alkyl. In another embodiment, R³⁶ is methyl or ethyl. In another embodiment, R³⁶ is methyl. In another embodiment, R³⁶ is ethyl.

In another embodiment, the compound for use in the compositions and methods provided herein has the formula:

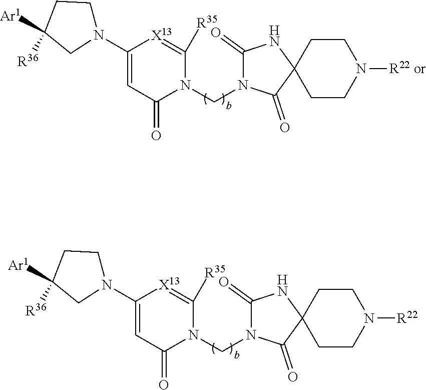

where the variables are as defined herein.

In another embodiment, the compound for use in the compositions and methods provided herein has Formula IIf:

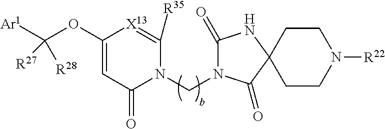

or a pharmaceutically acceptable derivative thereof, wherein:
b is 2 or 3;
X¹³ is N or CH;
Ar¹ is aryl;
R²² is heteroaryl;
R³⁵ is alkyl; and
R²⁷ and R²¹ are each independently H, alkyl or aralkyl, or together with the carbon atom to which they are attached form cycloalkyl.

In another embodiment, R²⁷ is alkyl and R²⁸ is H. In another embodiment, R²⁷ is methyl and R²⁸ is H. In another embodiment, R²⁷ and R²⁸ together with the carbon atom to which they are attached form cyclopropyl.

In another embodiment, the compound for use in the compositions and methods provided herein has the formula:

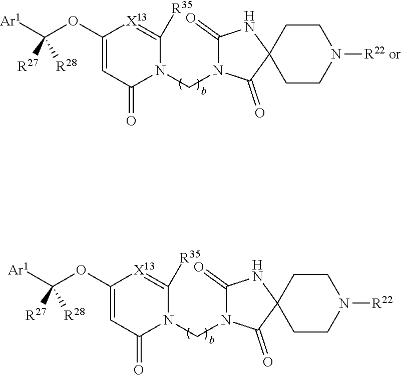

where the variables are as defined herein.

In another embodiment, c is 1, d and f are each 0, X¹² is a bond and the compound for use in the compositions and methods provided herein has Formula Ig:

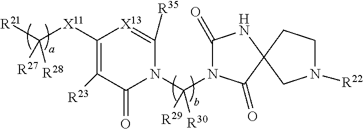

or a pharmaceutically acceptable derivative thereof, wherein:
b is an integer from 1-4;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl; and
R²³ to R²⁵, R²⁹, R³⁰ and R³⁵ are each independently H, alkyl or aralkyl; and
R²⁷ and R²¹ are each independently H, alkyl or aralkyl, or together with the carbon atom to which they are attached form cycloalkyl.

In another embodiment, each of R²³, R²⁹ and R³⁰ are H, c is 1, d and f are each 0, X¹² is a bond and the compound for use in the compositions and methods provided herein has Formula IIh:

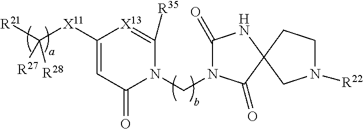

or a pharmaceutically acceptable derivative thereof, wherein:
b is an integer from 1-4;
a is an integer from 0-3;
X¹¹ is a bond, O, NR²⁵ or CO;
X¹³ is N or CH;
R²¹ is aryl, heteroaryl or heterocycloalkyl;
R²² is heteroaryl; and
R²⁵ and R³⁵ are each independently H or alkyl; and
R²⁷ and R²¹ are each independently H, alkyl or aralkyl, or together with the carbon atom to which they are attached form cycloalkyl.

In another embodiment, R²¹ in Formulae IIg or IIh is aryl or heterocycloalkyl. In another embodiment, R²¹ in Formulae IIg or IIh is aryl. In another embodiment, R²¹ in Formulae IIg or IIh is heterocycloalkyl.

In another embodiment, X¹¹ is a bond, each of R²³, R²⁹ and R³⁰ are H, c is 2, a, d and f are each 0, X¹² is a bond and the compound for use in the compositions and methods provided herein has Formula IIi:

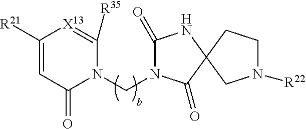

or a pharmaceutically acceptable derivative thereof, wherein:
b is 2 or 3;
X¹³ is N or CH;
R²¹ is heterocycloalkyl, optionally substituted with 1-3 substituents each independently selected from alkyl and aryl;
R²² is heteroaryl; and
R³⁵ is alkyl.

In another embodiment, the compound for use in the compositions and methods provided herein has Formula IIj:

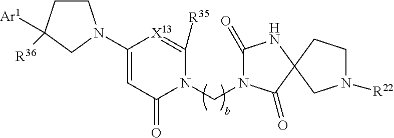

or a pharmaceutically acceptable derivative thereof, wherein:
b is 2 or 3;
X¹³ is N or CH;
Ar¹ is aryl;
R²² is heteroaryl;
R³⁵ is alkyl; and
R³⁶ is alkyl.

In another embodiment, R³⁶ is lower alkyl. In another embodiment, R³⁶ is methyl or ethyl. In another embodiment, R³⁶ is methyl. In another embodiment, R³⁶ is ethyl.

In another embodiment, the compound for use in the compositions and methods provided herein has the formula:

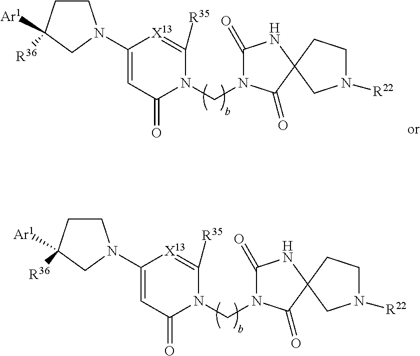

where the variables are as defined herein.

In another embodiment, the compound for use in the compositions and methods provided herein has Formula Ilk:

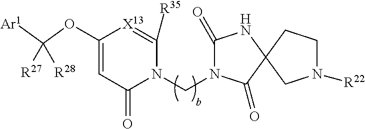

or a pharmaceutically acceptable derivative thereof, wherein:
b is 2 or 3;
X¹³ is N or CH;
Ar¹ is aryl;
R²² is heteroaryl;
R³⁵ is alkyl; and
R²⁷ and R²¹ are each independently H, alkyl or aralkyl, or together with the carbon atom to which they are attached form cycloalkyl.

In another embodiment, R²⁷ is alkyl and R²⁸ is H. In another embodiment, R²⁷ is methyl and R²⁸ is H. In another embodiment, R²⁷ and R²⁸ together with the carbon atom to which they are attached form cyclopropyl.

In another embodiment, the compound for use in the compositions and methods provided herein has the formula:

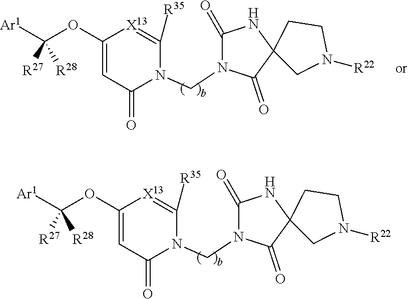

where the variables are as defined herein.

In another embodiment, b is 2. In another embodiment, b is 3.

In another embodiment, X¹³ is N. In another embodiment, X¹³ is CH.

In another embodiment, Ar¹ is unsubstituted aryl. In another embodiment, Ar¹ is optionally substituted phenyl. In another embodiment, Ar¹ is unsubstituted phenyl.

In another embodiment, R²² is bicyclic or monocyclic heteroaryl. In another embodiment, R²² is triazolopyrazinyl, imidazopyrazinyl, pyrazolopyrimidinyl, pyridyl, pyridazinyl or pyrimidinyl. In another embodiment, R²² is triazolopyrazinyl, imidazopyrazinyl, pyrazolopyrimidinyl or pyrimidinyl. In another embodiment, R²² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl or 2-pyrimidinyl. In another embodiment, R²² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl, 2-pyridyl, 3-pyridyl, 3-pyridazinyl or 2-pyrimidinyl. In another embodiment, R²² is [1,2,4]-triazolo[4,3-a]pyrazin-8-yl, imidazo[1,5-a]pyrazin-8-yl, 5-carboxyimidazo[1,5-a]pyrazin-8-yl, imidazo[1,2-a]pyrazin-8-yl, 1H-pyrazolo[3,4-d]pyrimidin-4-yl, 6-carboxy-3-pyridyl, 6-carboxy-2-pyridyl, 6-ethoxycarbonyl-2-pyridyl, 6-ethoxycarbonyl-3-pyridyl, 6-carboxy-3-pyridazinyl, 6-ethoxycarbonyl-3-pyridazinyl or 2-pyrimidinyl.

In another embodiment, R³⁵ is lower alkyl. In another embodiment, R³⁵ is methyl.

In one embodiment, provided herein is a compound for use in the compositions and methods provided herein having Formula IIm:

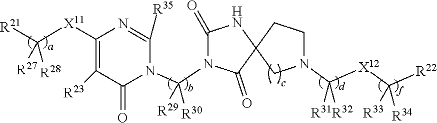

or a pharmaceutically acceptable derivative thereof, wherein
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
R²¹ is aryl, heteroaryl or heterocycloalkyl;
R²² is aryl or heteroaryl;
R²³, R²⁵, R²⁶ and R²⁹ to R³⁵ are each independently H, alkyl or aralkyl; and
R²⁷ and R²¹ are each independently H, alkyl or aralkyl, or together with the carbon atom to which they are attached form cycloalkyl.

In another embodiment, the compounds provided herein for use in the compositions and methods provided herein have Formula IIm, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁹, R³⁰ and R³⁵ are each independently H, alkyl or aralkyl; and
R²⁷ and R²¹ are each independently H, alkyl or aralkyl, or together with the carbon atom to which they are attached form cycloalkyl.

In another embodiment, the compounds provided herein for use in the compositions and methods provided herein have Formula IIm, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R² is heteroaryl;
R²³ and R²⁸ to R³⁰ are each independently H; and
R²⁷ and R³⁵ are each independently alkyl.

In another embodiment, the compounds provided herein for use in the compositions and methods provided herein have Formula IIm, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁹, R³⁰ and R³⁵ are each independently H, alkyl or aralkyl; and
R²⁷ and R²¹ are each independently H, alkyl or aralkyl, or together with the carbon atom to which they are attached form cycloalkyl.

In another embodiment, the compounds provided herein for use in the compositions and methods provided herein have Formula IIm, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R² is heteroaryl;
R²³ and R²⁸ to R³⁰ are each independently H; and
R²⁷ and R³⁵ are each independently alkyl.

In another embodiment, provided herein is a compound for use in the compositions and methods provided herein having Formula IIn:

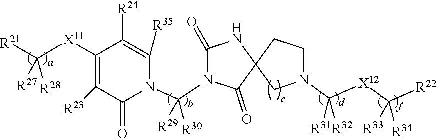

or a pharmaceutically acceptable derivative thereof, wherein
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
R²¹ is aryl, heteroaryl or heterocycloalkyl;
R²² is aryl or heteroaryl; and
R²³ to R²⁶ and R²⁹ to R³⁵ are each independently H, alkyl or aralkyl; and
R²⁷ and R²¹ are each independently H, alkyl or aralkyl, or together with the carbon atom to which they are attached form cycloalkyl.

In another embodiment, the compounds provided herein for use in the compositions and methods provided herein have Formula IIn, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴, R²⁹, R³⁰, and R³⁵ are each independently H, alkyl or aralkyl; and
R²⁷ and R²¹ are each independently H, alkyl or aralkyl, or together with the carbon atom to which they are attached form cycloalkyl.

In another embodiment, the compounds provided herein for use in the compositions and methods provided herein have Formula IIn, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴ and R²⁸ to R³⁰ are each independently H; and
R²⁷ and R³⁵ are each independently alkyl.

In another embodiment, the compounds provided herein for use in the compositions and methods provided herein have Formula IIn, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴, R²⁹, R³⁰, and R³⁵ are each independently H, alkyl or aralkyl; and
R²⁷ and R²¹ are each independently H, alkyl or aralkyl, or together with the carbon atom to which they are attached form cycloalkyl.

In another embodiment, the compounds provided herein for use in the compositions and methods provided herein have Formula IIn, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴ and R²⁸ to R³⁰ are each independently H; and
R²⁷ and R³⁵ are each independently alkyl.

In another embodiment, the compound for use in the compositions and methods provided herein has Formula IIo:

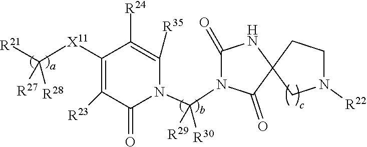

wherein:
b is an integer from 1-4;
c is an integer from 1-3;
a is an integer from 0-3;
X¹¹ is a bond, O, NR²⁵ or CO;
R²¹ is aryl or heterocycloalkyl;
R²² is heteroaryl; and
R²³ to R²⁵, R²⁷ to R³⁰ and R³⁵ are each independently H or alkyl.

In another embodiment, the compound for use in the compositions and methods provided herein has Formula IIp:

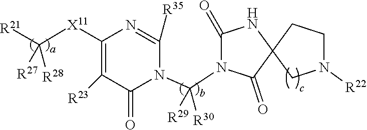

wherein:
b is an integer from 1-4;
c is an integer from 1-3;
a is an integer from 0-3;
X¹¹ is a bond, O, NR²⁵ or CO;
R²¹ is aryl or heterocycloalkyl;
R²² is heteroaryl; and
R²³, R²⁵, R²⁷ to R³⁰ and R³⁵ are each independently H or alkyl.

In another embodiment, the compounds for use in the compositions and methods provided herein have Formula Ho or IIp, wherein:
b is 2 or 3;
c is 2;
a is 0 or 1;
X¹¹ is a bond or O;
R²¹ is aryl or heterocycloalkyl, each optionally substituted with 1-3 substituents each independently selected from alkyl and aryl;
R²² is heteroaryl; and
R²³, R²⁵, R²⁷ to R³⁰ and R³⁵ are each independently H or alkyl.

In another embodiment, the compounds for use in the compositions and methods provided herein have Formula Ho or IIp, wherein:
b is 2 or 3;
c is 1;
a is 0 or 1;
X¹¹ is a bond or O;
R²¹ is aryl or heterocycloalkyl, each optionally substituted with 1-3 substituents each independently selected from alkyl and aryl;
R²² is heteroaryl; and
R²³, R²⁵, R²⁷ to R³⁰ and R³⁵ are each independently H or alkyl.

In one embodiment, the compounds provided herein for use in the compositions and methods provided herein are those shown in Table 2 below:

| Compound | Structure |
|---|---|
| 25 | 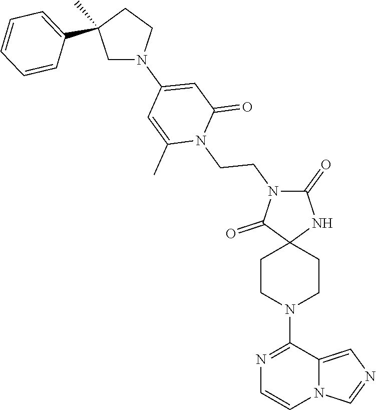 [SMILES: Cc1cc(N2CC[C@](C)(c3ccccc3)C2)cc(=O)n1CCN1C(=O)NC2(CCN(c3nccn4cncc34)CC2)C1=O] |
| 26 | 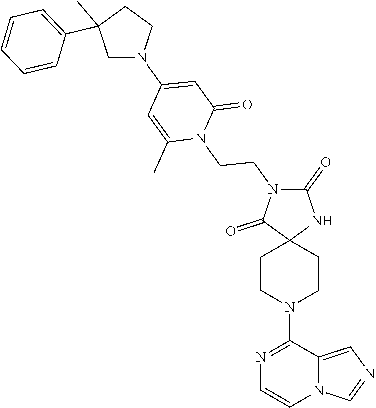 [SMILES: Cc1cc(N2CCC(C)(c3ccccc3)C2)cc(=O)n1CCN1C(=O)NC2(CCN(c3nccn4cncc34)CC2)C1=O] |
| 27 | 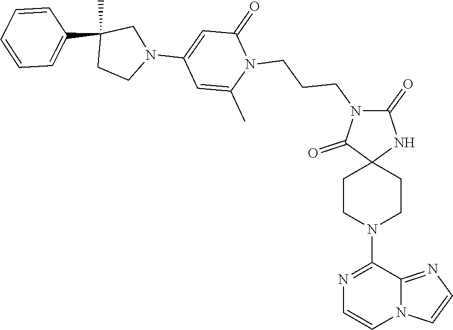 [SMILES: Cc1cc(N2CC[C@@](C)(c3ccccc3)C2)cc(=O)n1CCCN1C(=O)NC2(CCN(c3nccn4ccnc34)CC2)C1=O] |
| 28 | 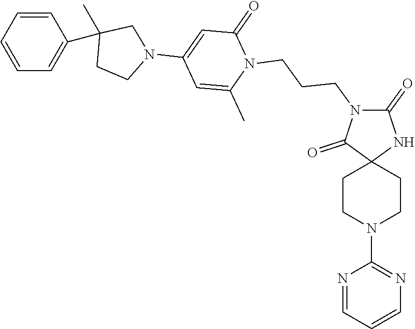 [SMILES: Cc1cc(N2CCC(C)(c3ccccc3)C2)cc(=O)n1CCCN1C(=O)NC2(CCN(c3ncccn3)CC2)C1=O] |
| 29 | 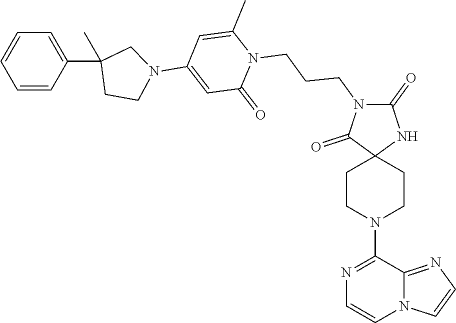 [SMILES: Cc1cc(N2CCC(C)(c3ccccc3)C2)cc(=O)n1CCCN1C(=O)NC2(CCN(c3nccn4ccnc34)CC2)C1=O] |
| 30 | 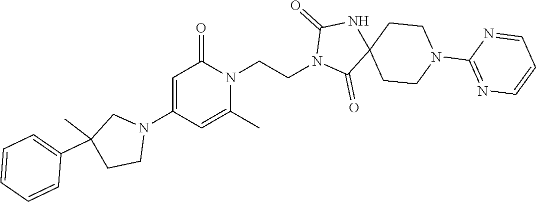 [SMILES: Cc1cc(N2CCC(C)(c3ccccc3)C2)cc(=O)n1CCN1C(=O)NC2(CCN(c3ncccn3)CC2)C1=O] |
| 31 | 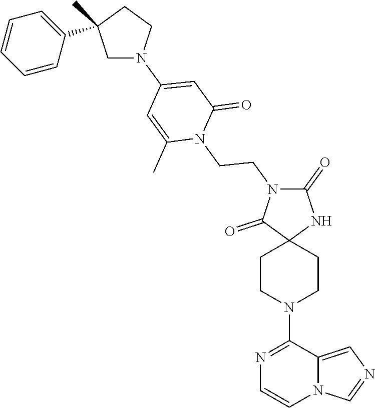 [SMILES: Cc1cc(N2CC[C@@](C)(c3ccccc3)C2)cc(=O)n1CCN1C(=O)NC2(CCN(c3nccn4cncc34)CC2)C1=O] |
| 32 | 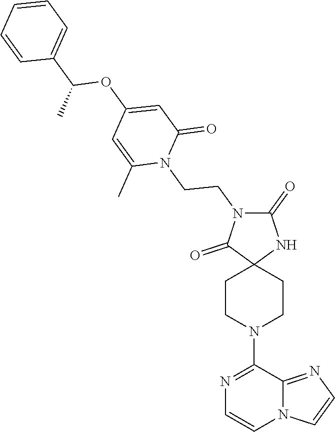 [SMILES: Cc1cc(O[C@H](C)c2ccccc2)cc(=O)n1CCN1C(=O)NC2(CCN(c3nccn4ccnc34)CC2)C1=O] |
| 33 | 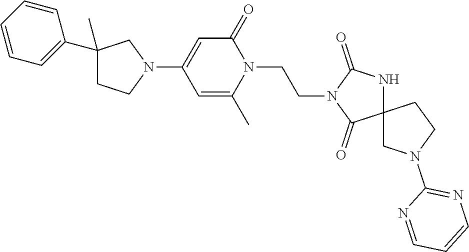 [SMILES: Cc1cc(N2CCC(C)(c3ccccc3)C2)cc(=O)n1CCN1C(=O)NC2(CCN(c3ncccn3)C2)C1=O] |
| 34 | 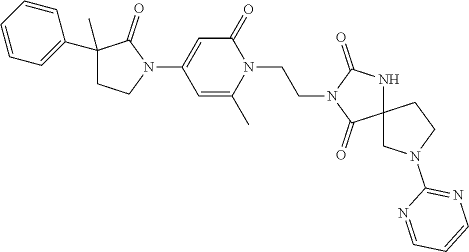 [SMILES: Cc1cc(N2CCC(C)(c3ccccc3)C2=O)cc(=O)n1CCN1C(=O)NC2(CCN(c3ncccn3)C2)C1=O] |
| 35 | 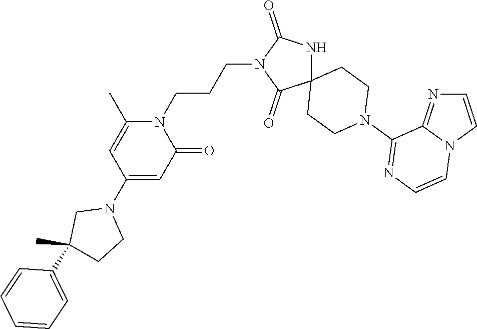 [SMILES: Cc1cc(N2CC[C@](C)(c3ccccc3)C2)cc(=O)n1CCCN1C(=O)NC2(CCN(c3nccn4ccnc34)CC2)C1=O] |

### III. Synthesis of the Compounds

The compounds provided herein may be prepared according to synthetic methods well known to those of skill in the art. In one embodiment, the compounds may be prepared according to Scheme I:

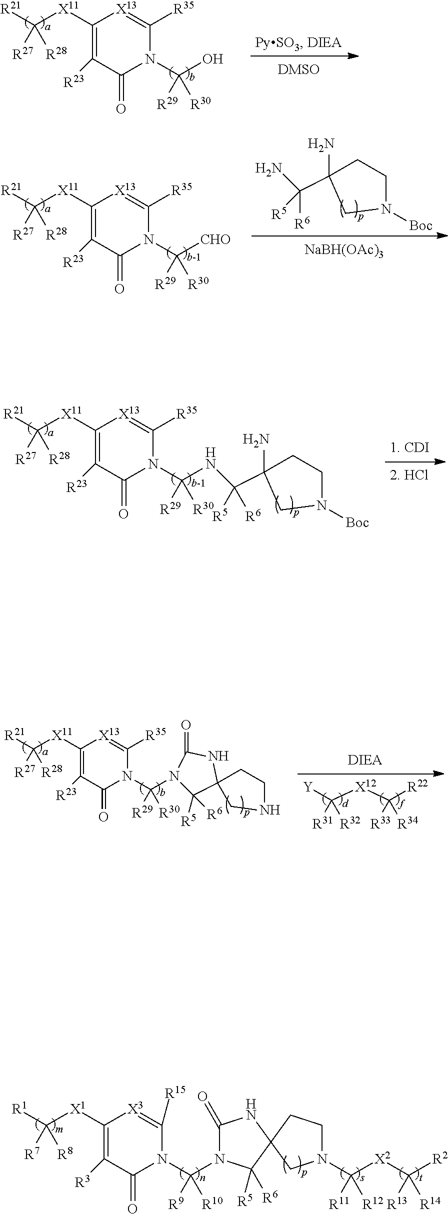

where Y is a leaving group.

In another embodiment, the compounds may be prepared according to Scheme IL:

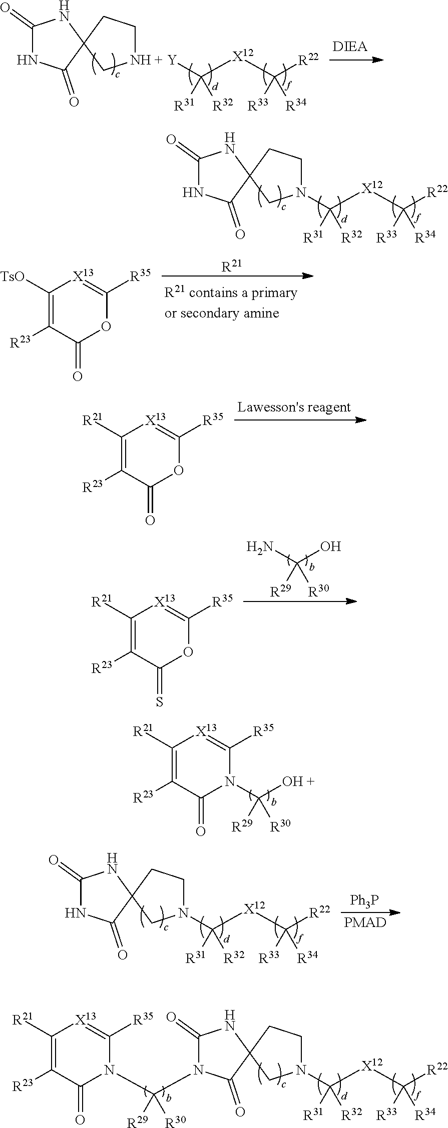

where Y is a leaving group.

In another embodiment, the compounds may be prepared according to Scheme III:

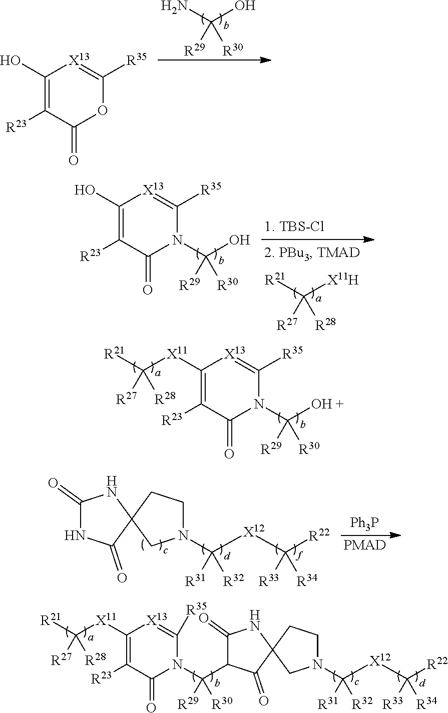

### IV. Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compounds remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### A. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, crospovidone, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### B. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.10% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### C. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### D. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracistemal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### E. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### F. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### G. Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### H. Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof; or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### V. Dosing

The compounds and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

### VI. Methods of Treatment

Free light chains (FLCs) adopt a well-defined homodimeric structure, wherein the monomers may be covalently linked by an interchain disulfide bond. LC monomers comprise an N-terminal variable (V) domain attached to a C-terminal constant (C) domain. Amyloidogenic FLCs involved in AL patients, are less stable than non-amyloidogenic FLCs, can misfold and misassemble into nonnative species including cross-β-sheet amyloid fibrils, a hallmark of AL amyloidosis. The structure-proteotoxicity relationship is not fully understood but several processes have been described, including destabilization-dependent endoproteolysis that releases amyloidogenic LC fragments. LC fragments including V domains are observed in patient deposits alongside full length LCs. Without being bound by any theory, it is believed that the compounds and compositions provided herein stop light chain conformational excursions at the beginning of the aggregation cascade via kinetic stabilization of FLCs. It is also believed that the compounds provided herein bind to conserved regions of LCs, thereby allowing the compounds to stabilize many immunoglobulin light chains and to be effective in many AL subjects.

Kinetic stabilization of LCs is unlikely to contribute to plasma cell death but could reduce organ proteotoxicity and the progression of AL. Subjects with prominent cardiac involvement currently have few available options for treatment and represent an urgent unmet medical need, as they are often too sick to tolerate chemotherapy. Reduction of organ proteotoxicity could allow the subject to tolerate chemotherapy. Thus, in one embodiment, provided herein is a method of pretreatment of a subject having AL with prominent cardiac involvement with a compound provided herein, followed by chemotherapy.

In another embodiment, provided herein is a method of treating light chain amyloidosis by administering to a subject a compound or composition provided herein. In one embodiment, the subject has light chain amyloidosis and is treatment naïve. In another embodiment, the subject has relapsed or refractory light chain amyloidosis.

In another embodiment, provided herein is a method of stabilizing immunoglobulin light chains by contacting the immunoglobulin light chains with a compound provided herein. In one embodiment, the immunoglobulin light chains are stabilized in a native conformation thereof. In certain embodiments, the immunoglobulin light chains are dimers. Thus, in certain embodiments, provided herein is a method of stabilizing immunoglobulin light chain dimers in a native conformation. As used herein, “native conformation” refers to a conformation of immunoglobulin light chains present in subjects not having light chain amyloidosis.

In another embodiment, provided herein is a method of preventing or lessening immunoglobulin light chain misfolding and/or endoproteolysis by contacting the immunoglobulin light chains with a compound provided herein.

In another embodiment, provided is a method of maintenance therapy upon recurrence of light chain amyloidosis following primary treatment by administering to a subject a compound or composition provided herein. In such embodiments, reemergence of the clonal plasma cells is generally slow, and thus organ toxicity caused by conformationally unstable circulating LC can be minimized by kinetic stabilizer treatment.

### VII. Combination Therapy with a Second Active Agent

The compound provided herein, or a derivative thereof, can also be combined or used in combination with other therapies or therapeutic agents useful in the treatment and/or prevention of light chain amyloidosis.

In one embodiment, provided herein is a method of treating, preventing, or managing light chain amyloidosis, comprising administering to a subject a compound provided herein, or a derivative thereof, in combination with one or more second active agents. In one embodiment, the second active agent is a proteasome inhibitor (e.g., bortezomib, ixazomib, carfilzomib). In another embodiment, the second active agent is a chemotherapeutic agent, including but not limited to alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129 (sorrentotherapeutics.com)) and/or an anti-amyloid antibody. In another embodiment, the compounds provided herein, or a derivative thereof, are used in combination with stem cell transplant therapy. In further embodiments, the second active agent is selected from those disclosed in PCT Publication Nos. WO 2020/205683, WO 2019/191558, WO 2017/117430 or WO 2021/007594. In another embodiment, the second active agent is a therapeutic agent that promotes clearance of amyloid deposits, such as CAEL-101 (caelumbio.com) or NEOD001 (birtamimab).

In one embodiment, the second active agent is a plasma cell directed therapy, including high-dose cyclophosphamide combined with an anti-thymocyte antibody (e.g., Thymoglobulin®, Atgam®)(with subsequent autologous stem cell transplantation), an immunomodulatory agent (e.g., lenalidomide), a steroid (e.g., dexamethasone), a proteasome inhibitor (e.g., bortezomib), atacicept, or an anti-CD38 antibody (e.g., daratumumab, isatuximab). In another embodiment, the second active agent is a combination of daratumumab, bortezomib, cyclophosphamide and dexamethasone.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, a compound provided herein, e.g., the compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more second active ingredients or agents can be used together with the compound provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins.

In one embodiment, the compound provided herein, or a derivative thereof, can be administered in an amount ranging from about 0.1 to about 150 mg, from about 1 to about 25 mg, or from about 2 to about 10 mg orally and daily alone, or in combination with a second active agent, prior to, during, or after the use of conventional therapy.

### VIII. Examples

The examples below are meant to illustrate certain embodiments provided herein, and not to limit the scope of this disclosure.

### A. Analytical Conditions

1H NMR Conditions

Instrument Type: AVANCE III 400 or AVANCE III 400 HD or AVANCE NEO; Probe Type: 5 mm PABBO BB or 5 mm CPP BBO; Frequency (MHz): 400.1300; Temperature (Degree ° C.): 27.

LCMS Methods

Method 1:

Instrument: SHIMADZU LCMS-2020; Column: Kinetex EVO C18 2.1×30 mm, 5 μm; Mobile Phase: A: 0.0375% TFA in water (v/v), B: 0.01875% TFA in Acetonitrile (v/v); Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

Method 2:

Instrument: SHIMADZU LCMS-2020; Column: Kinetex EVO C18 2.1×30 mm, 5 μm; Mobile Phase: A: 0.025% NH₃·H₂O in water (v/v), B: Acetonitrile; Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

HPLC Methods

Method 1:

Instrument: SHIMADZU LC-20AB; Column: Kinetex C18 LC Column 4.6×50 mm, 5 μm; Mobile Phase: A: 0.0375% TFA in water (v/v), B: 0.01875% TFA in Acetonitrile (v/v); Gradient: 0.0 min 10% B→2.40 min 80% B→3.70 min 80% B→3.71 min 10% B→4.00 min 10% B; Flow: 1.5 mL/min; Column Temp: 50° C.; Detector: PDA (220 nm & 215 nm & 254 nm).

Method 2:

Instrument: SHIMADZU LC-20AB; Column: XBridge C18, 2.1×50 mm, 5 μm; Mobile Phase: A: 0.025% NH₃·H₂O in water (v/v), B: Acetonitrile; Gradient: 0.0 min 10% B→4.20 min 80% B→5.30 min 80% B→5.31 min 10% B→6.00 min 10% B; Flow: 0.8 mL/min; Column Temp: 40° C.; Detector: PDA (220 nm & 215 nm & 254 nm).

Method 3:

Instrument: SHIMADZU LC-20AB; Column: XBridge C18, 2.1×50 mm, 3.5 μm; Mobile Phase: A: 0.025% NH₃·H₂O in water (v/v), B: Acetonitrile; Gradient: 0.0 min 30% B→3.00 min 90% B→3.50 min 90% B→3.51 min 30% B→4.00 min 30% B; Flow: 1.2 mL/min; Column Temp: 50° C.; Detector: PDA (220 nm & 215 nm & 254 nm).

### Example 1

### Compound 28: 3-(3-(6-methyl-4-(3-methyl-3-phenylpyrrolidin-1-yl)-2-oxopyridin-1(2H)-yl)propyl)-8-(pyrimidin-2-yl)-1,3,8-triazaspiro[4.5]decane-2,4-dione

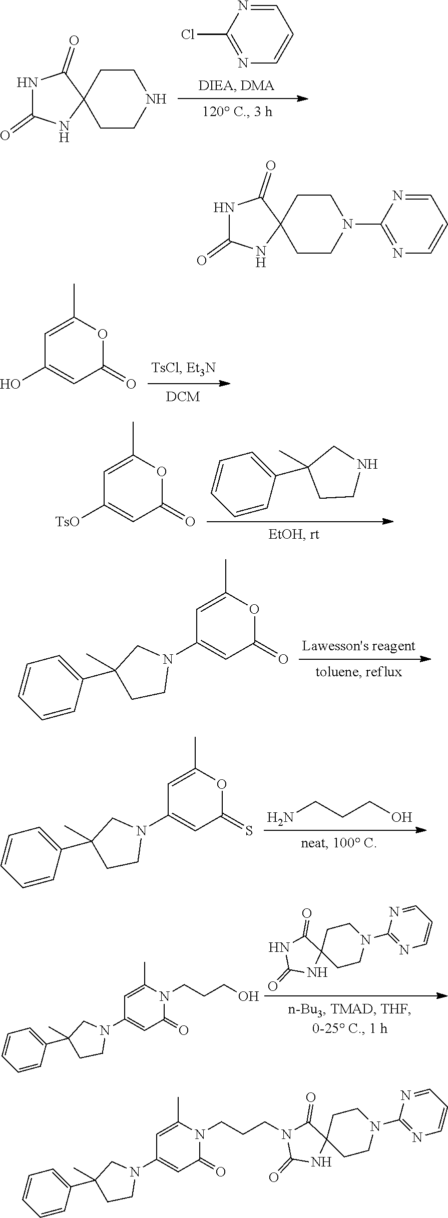

### Step 1: 8-(pyrimidin-2-yl)-1,3,8-triazaspiro[4.5]decane-2,4-dione

To a mixture of 1,3,8-triazaspiro[4.5]decane-2,4-dione (600 mg, 3.55 mmol, 1 equiv) and 2-chloropyrimidine (406 mg, 3.55 mmol, 1 equiv) in DMA (8 mL) was added DIEA (1.83 g, 14.19 mmol, 4 equiv), then the mixture was stirred at 120° C. for 3 h. The reaction mixture was diluted with water 40 mL and extracted with EtOAc (40 mL×2). The combined organic layers were washed with brine (40 mL), dried, filtered and concentrated to give a residue, which was purified by prep-HPLC (FA condition: column: Phenomenex luna C18 150*25 mm*10 μm; mobile phase: [water(FA)-ACN]; B %: 28%-58%, 10 min) to afford the title compound (800 mg, 91%) as a yellow solid.

LC-MS [ESI, M+1]: 247.7.

### Step 2: 6-methyl-2-oxo-2H-pyran-4-yl 4-methylbenzenesulfonate

To a solution of 4-hydroxy-6-methyl-pyran-2-one (10.00 g, 79 mmol, 1 equiv), Et₃N (16.00 g, 159 mmol, 2 equiv) in DCM (250 mL) was added TsCl (6.15 g, 87 mmol, 1.1 equiv) dropwise at 0° C. The mixture was stirred at 25° C. for 12 h. The reaction mixture was diluted with water (100 mL), extracted with DCM (100 mL×3), the combined organic layers were washed with brine (50 mL), dried, filtered and concentrated to give the title compound (19.83 g, 89%) as a white solid.

¹H NMR (400 MHz, CDCl₃): δ 7.74 (d, J=8.0 Hz, 2H), 7.32 (d, J=8.0 Hz, 2H), 5.92 (s, 1H), 5.75 (s, 1H), 2.40 (s, 3H), 2.16 (s, 3H).

LC-MS [ESI, M+1]: 281.4.

### Step 3: 6-methyl-4-(3-methyl-3-phenylpyrrolidin-1-yl)-2H-pyran-2-one

To a solution of 6-methyl-2-oxo-2H-pyran-4-yl 4-methylbenzenesulfonate (5.00 g, 17.80 mmol, 1 equiv) in EtOH (10 mL) was added 3-methyl-3-phenylpyrrolidine (3.16 g, 19.60 mmol, 1.1 equiv). The mixture was stirred at 80° C. for 12 h. The reaction mixture was filtered and concentrated under reduced pressure to give a residue, which was purified by flash silica gel chromatography (ISCO®; 20 g Sepa Flash® Silica Flash Column, Eluent of 0-10% Dichloromethane:Methanol @ 45 mL/min) to give the title compound (2.20 g, 45%) as a yellow gum.

¹H NMR (400 MHz, CDCl₃): δ 7.40-7.38 (m, 2H), 7.30 (d, J=1.2 Hz, 1H), 7.26 (s, 2H), 5.75 (d, J=13.6 Hz, 1H), 4.95 (d, J=8.4 Hz, 1H), 3.69-3.44 (m, 4H), 2.38-2.31 (m, 1H), 2.23 (s, 4H), 1.44 (s, 3H).

LC-MS [ESI, M+1]: 270.2.

### Step 4: 6-methyl-4-(3-methyl-3-phenylpyrrolidin-1-yl)-2H-pyran-2-thione

A mixture of 6-methyl-4-(3-methyl-3-phenylpyrrolidin-1-yl)-2H-pyran-2-one (2.60 g, 9.65 mmol, 1 equiv) and 2,4-bis(4-methoxyphenyl)-2,4-dithioxo-1,3,2λ5,4λ5-dithiadiphosphetane (5.86 g, 14.48 mmol, 1.5 equiv) in toluene (10 mL) was degassed and purged with N₂ for 3 times, and then the mixture was stirred at 90° C. for 12 h. The reaction mixture was concentrated under reduced pressure to give a residue, which was purified by flash silica gel chromatography (ISCO®; 20 g Sepa Flash® Silica Flash Column, Eluent of 0˜10% Dichloromethane:Methanol @ 45 mL/min) to give the title compound (2.00 g, 56%) as a yellow solid.

¹H NMR (400 MHz, CDCl₃): δ 7.39-7.35 (m, 2H), 7.29 (d, J=7.6 Hz, 1H), 7.26-7.21 (m, 2H), 6.46-6.39 (m, 1H), 5.97-5.92 (m, 1H), 3.73-3.69 (m, 1H), 3.63-3.49 (m, 3H), 2.38-2.32 (m, 4H), 2.28-2.25 (m, 1H), 1.42 (d, J=2.0 Hz, 3H).

LC-MS [ESI, M+1]: 285.9.

### Step 5: 1-(3-hydroxypropyl)-6-methyl-4-(3-methyl-3-phenylpyrrolidin-1-yl)pyridin-2(1H)-one

A mixture of 6-methyl-4-(3-methyl-3-phenylpyrrolidin-1-yl)-2H-pyran-2-thione (200 mg, 0.7 mmol, 1 equiv) and 3-aminopropan-1-ol (526 mg, 7.01 mmol, 10 equiv) was stirred at 100° C. for 2 h. The mixture was concentrated under the reduced pressure to give a residue, which was purified by prep-HPLC (FA condition: column: Phenomenex luna C18 150*25 mm*10 μm; mobile phase: (water (FA)-ACN); B %: 27%-57%, 10 min) to give the title compound (65 mg, 28%) as a yellow solid.

¹H NMR (400 MHz, CDCl₃): δ 8.25 (s, 1H), 7.39-7.35 (m, 2H), 7.30-7.26 (m, 3H), 5.72 (d, J=2.0 Hz, 1H), 5.52 (d, J=2.4 Hz, 1H), 4.78-4.41 (m, 1H), 4.21-4.18 (m, 2H), 3.62-3.42 (m, 6H), 2.38 (s, 3H), 2.39-2.29 (m, 1H), 2.24-2.11 (m, 1H), 1.85 (m, 2H), 1.42 (s, 3H).

LC-MS [ESI, M+1]: 327.6.

### Step 6: 3-(3-(6-methyl-4-(3-methyl-3-phenylpyrrolidin-1-yl)-2-oxopyridin-1(2H)-yl)propyl)-8-(pyrimidin-2-yl)-1,3,8-triazaspiro[4.5]decane-2,4-dione

To a solution of TMAD (16 mg, 92 μmol, 1.5 equiv) in THF (0.5 mL) was added tributylphosphane (19 mg, 92 μmol, 1.5 equiv) and the mixture was stirred at 0° C. for 0.5 h, then 1-(3-hydroxypropyl)-6-methyl-4-(3-methyl-3-phenylpyrrolidin-1-yl)pyridin-2(1H)-one (20 mg, 61 μmol, 1 equiv) and 8-(pyrimidin-2-yl)-1,3,8-triazaspiro[4.5]decane-2,4-dione (15 mg, 61 μmol, 1 equiv) was added to the reaction mixture and stirred at 25° C. for another 1 h.

The reaction mixture was concentrated under the reduced pressure. The residue was purified by prep-HPLC (FA condition: column: Phenomenex luna C18 150*25 mm*10 μm; mobile phase: (water (FA)-ACN); B %: 35%-65%, 10 min) to give the title compound (3 mg, 8%) as a yellow solid.

¹H NMR (400 MHz, CDCl₃): δ 8.23 (d, J=4.8 Hz, 2H), 7.29-7.17 (m, 2H), 7.19 (s, 3H), 6.77 (s, 1H), 6.43 (t, J=4.4 Hz, 1H), 5.52 (s, 1H), 5.33 (s, 1H), 4.49 (td, J=14.0, 4.0 Hz, 2H), 3.93-3.90 (m, 2H), 3.55 (t, J=6.4 Hz, 2H), 3.49-3.31 (m, 6H), 2.23 (s, 3H), 2.20-2.14 (m, 1H), 2.06-2.01 (m, 3H), 1.95-1.90 (m, 2H), 1.67 (d, J=13.6 Hz, 2H), 1.31 (s, 3H).

LC-MS [ESI, M+1]: 556.1.

DSF: **** (see Example 6)

| Compound | | | |
|---|---|---|---|
| Number | Synthesis | LC/MS | DSF¹ |
| Compound 25 | Similar to Example 1 | m/z 581.2 [M + H]⁺ | ****** |
| Compound 26 | Similar to Example 1 | m/z 581.4 [M + H]⁺ | ****** |
| Compound 27 | Similar to Example 1 | m/z 595.2 [M + H]⁺ | **** |
| Compound 29 | Similar to Example 1 | m/z 595.2 [M + H]⁺ | **** |
| Compound 30 | Similar to Example 1 | m/z 542.2 [M + H]⁺ | **** |
| Compound 31 | Similar to Example 1 | m/z 581.5 [M + H]⁺ | *** |
| Compound 35 | Similar to Example 1 | m/z 595.0 [M + H]⁺ | *** |

¹See Example 6

### Example 2

### Compound 1: (S)-8-([1,2,4]triazolo[4,3-a]pyrazin-8-yl)-3-(2-(4-(3-ethyl-3-phenylpyrrolidin-1-yl)-6-methyl-2-oxopyridin-1(2H)-yl)ethyl)-1,3,8-triazaspiro[4.5]decan-2-one

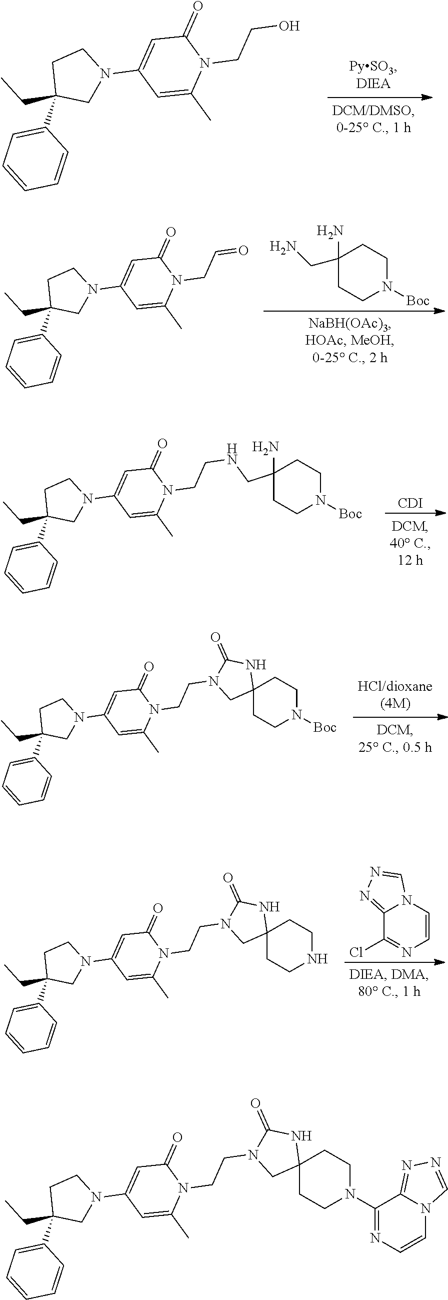

### Step 1: (S)-2-(4-(3-ethyl-3-phenylpyrrolidin-1-yl)-6-methyl-2-oxopyridin-1(2H)-yl)acetaldehyde

To a solution of 4-[(3S)-3-ethyl-3-phenyl-pyrrolidin-1-yl]-1-(2-hydroxyethyl)-6-methyl-pyridin-2-one (200 mg, 0.61 mmol, 1 equiv) and DIEA (238 mg, 1.84 mmol, 3 equiv) in DCM (4 mL) was added a solution of pyridine; sulfur trioxide (146 mg, 0.92 mmol, 1.5 equiv) in DMSO (2 mL) dropwise at 0° C. The mixture was stirred at 25° C. for 1 h. The reaction was quenched by H₂O (10 mL) and then extracted with DCM (15 mL×5). The combined organic phase was washed with brine (15 mL×5), dried over sodium sulfate, filtered, and concentrated to give a residue. The residue was purified by flash silica gel chromatography (ISCO®; 12 g SepaFlash® Silica Flash Column, Eluent of 0˜3% DCM/MeOH @ 65 mL/min) to afford the title compound (120 mg, 60%) as a light-yellow oil.

LC-MS [ESI, M+1]: 325.2.

### Step 2: tert-butyl (S)-4-amino-4-(((2-(4-(3-ethyl-3-phenylpyrrolidin-1-yl)-6-methyl-2-oxopyridin-1(2H)-yl)ethyl)amino)methyl)piperidine-1-carboxylate

To a solution of 2-[4-[(3S)-3-ethyl-3-phenyl-pyrrolidin-1-yl]-2-methyl-6-oxo-1-pyridyl]acetaldehyde (110 mg, 0.34 mmol, 1 equiv), tert-butyl 4-amino-4-(aminomethyl)piperidine-1-carboxylate (78 mg, 0.34 mmol, 1 equiv) and HOAc (21 mg, 0.34 mmol, 1 equiv) in MeOH (2.5 mL) was added NaBH₃CN (64 mg, 1.02 mmol, 3 equiv) at 0° C. The mixture was stirred at 0° C. for 1 h and then stirred at 25° C. for 1 h. The reaction mixture was concentrated. The residue was diluted with water (10 mL) and then extracted with DCM (15 mL×3). The combined organic phase was washed with brine (15 mL×3), dried over sodium sulfate, filtered, and concentrated to give a residue. The residue was purified by prep-TLC (Dichloromethane:Methanol=10:1, R_{f}=0.2) to afford the title compound (100 mg, 55%) as a yellow oil.

LC-MS [ESI, M+1]: 538.3.

### Step 3: tert-butyl (S)-3-(2-(4-(3-ethyl-3-phenylpyrrolidin-1-yl)-6-methyl-2-oxopyridin-1(2H)-yl)ethyl)-2-oxo-1,3,8-triazaspiro[4.5]decane-8-carboxylate

To a solution of tert-butyl (S)-4-amino-4-(((2-(4-(3-ethyl-3-phenylpyrrolidin-1-yl)-6-methyl-2-oxopyridin-1(2H)-yl)ethyl)amino)methyl)piperidine-1-carboxylate (100 mg, 0.18 mmol, 1 equiv) in DCM (6 mL) was added CDI (241 mg, 1.49 mmol, 8 equiv). The reaction mixture was stirred at 40° C. for 12 h. The reaction mixture was diluted with water (10 mL) and adjusted to pH 8 with NaHCO₃ (aq.). The aqueous solution was extracted with DCM (15 mL×3). The combined organic phase was washed with brine (15 mL×3), dried over sodium sulfate, filtered, and concentrated to give a residue. The residue was purified by prep-TLC (SiO₂, DCM:MeOH=15:1, Rf=0.3) to afford the title compound (85 mg, 81%) as a white solid.

¹H NMR (400 MHz, CDCl3) δ 7.40-7.31 (m, 2H), 7.27-7.23 (m, 1H), 7.20 (d, J=7.2 Hz, 2H), 5.63 (s, 1H), 5.40 (s, 1H), 4.70 (s, 1H), 4.14 (t, J=6.4 Hz, 2H), 3.65-3.58 (m, 1H), 3.51 (t, J=6.8 Hz, 5H), 3.41 (s, 1H), 3.37-3.26 (m, 5H), 2.39 (s, 3H), 2.33-2.20 (m, 2H), 1.89-1.79 (m, 1H), 1.67-1.57 (m, 5H), 1.46 (s, 9H), 0.70 (t, J=7.2 Hz, 3H).

LC-MS [ESI, M+1]: 564.3.

### Step 4: (S)-3-(2-(4-(3-ethyl-3-phenylpyrrolidin-1-yl)-6-methyl-2-oxopyridin-1(2H)-yl)ethyl)-1,3,8-triazaspiro[4.5]decan-2-one

To a solution of tert-butyl (S)-3-(2-(4-(3-ethyl-3-phenylpyrrolidin-1-yl)-6-methyl-2-oxopyridin-1(2H)-yl)ethyl)-2-oxo-1,3,8-triazaspiro[4.5]decane-8-carboxylate (80 mg, 0.14 mmol, 1 equiv) in DCM (2 mL) was added HCl/dioxane (4 M, 1 mL, 28 equiv). The reaction mixture was stirred at 25° C. for 0.5 h. The reaction mixture was concentrated under the reduced pressure to afford the title compound (65 mg, 92%) as a white solid.

¹H NMR (400 MHz, CDCl₃ δ 9.80-9.21 (m, 1H), 9.00 (s, 1H), 7.72-7.45 (m, 1H), 7.32 (s, 2H), 7.22 (s, 3H), 6.64-6.09 (m, 2H), 4.40-4.12 (m, 2H), 3.73-3.27 (m, 12H), 2.70-2.54 (m, 3H), 2.32 (s, 2H), 2.22-1.94 (m, 4H), 1.86-1.65 (m, 2H), 0.67 (s, 3H).

LC-MS [ESI, M+1]: 464.1.

### Step 5: (S)-8-([1,2,4]triazolo[4,3-a]pyrazin-8-yl)-3-(2-(4-(3-ethyl-3-phenylpyrrolidin-1-yl)-6-methyl-2-oxopyridin-1(2H)-yl)ethyl)-1,3,8-triazaspiro[4.5]decan-2-one

To a solution of (S)-3-(2-(4-(3-ethyl-3-phenylpyrrolidin-1-yl)-6-methyl-2-oxopyridin-1(2H)-yl)ethyl)-1,3,8-triazaspiro[4.5]decan-2-one (30 mg, 60 μmol, 1 equiv, HCl) and 8-chloro-[1,2,4]triazolo[4,3-a]pyrazine 7 (9 mg, 60 μmol, 1 equiv) in DMA (1 mL) was added DIEA (39 mg, 0.30 mmol, 5 equiv). The reaction mixture was stirred at 80° C. for 1 h.

The reaction mixture was purified by prep-HPLC (column: Waters xbridge 150*25 mm 10 μm; mobile phase: (water(NH₄HCO₃)-ACN); B %: 24%-54%, 9 min) to afford the title compound (15 mg, 43%) as a white solid.

¹H NMR (400 MHz, CDCl₃): δ 8.72 (s, 1H), 7.43 (d, J=4.4 Hz, 1H), 7.33-7.28 (m, 3H), 7.24-7.17 (m, 3H), 5.64 (s, 1H), 5.39 (s, 1H), 4.87 (s, 1H), 4.54 (d, J=4.0 Hz, 2H), 4.14 (t, J=6.4 Hz, 4H), 3.63-3.49 (m, 4H), 3.37 (s, 2H), 2.38 (s, 3H), 2.31-2.18 (m, 2H), 1.88-1.81 (m, 3H), 1.70-1.63 (m, 3H), 1.33-1.25 (m, 2H), 0.66 (t, J=7.2 Hz, 3H).

LC-MS [ESI, M+1]: 582.4.

DSF: ***** (see Example 6)

| Compound | | | |
|---|---|---|---|
| Number | Synthesis | LC/MS | DSF¹ |
| Compound 2 | Similar to Example 2 | m/z 581.2 [M + H]⁺ | ****** |
| Compound 3 | Similar to Example 2 | m/z 567.2 [M + H]⁺ | ****** |
| Compound 4 | Similar to Example 2 | m/z 568.1 [M + H]⁺ | ****** |
| Compound 5 | Similar to Example 2 | m/z 567.3 [M + H]⁺ | ****** |
| Compound 6 | Similar to Example 2 | m/z 581.4 [M + H]⁺ | ****** |
| Compound 7 | Similar to Example 2 | m/z 528.4 [M + H]⁺ | ****** |
| Compound 8 | Similar to Example 2 | m/z 567.5 [M + H]⁺ | ****** |
| Compound 9 | Similar to Example 2 | m/z 568.2 [M + H]⁺ | **** |
| Compound 10 | Similar to Example 2 | m/z 581.3 [M + H]⁺ | **** |
| Compound 14 | Similar to Example 2 | m/z 613.4 [M + H]⁺ | ***** |
| Compound 15 | Similar to Example 2 | m/z 581.4 [M + H]⁺ | **** |
| Compound 16 | Similar to Example 2 | m/z 581.5 [M + H]⁺ | **** |
| Compound 17 | Similar to Example 2 | m/z 586.3 [M + H]⁺ | ***** |
| Compound 18 | Similar to Example 2 | m/z 600.4 [M + H]⁺ | ***** |
| Compound 19 | Similar to Example 2 | m/z 585.4 [M + H]⁺ | ** |
| Compound 20 | Similar to Example 2 | m/z 585.4 [M + H]⁺ | **** |
| Compound 21 | Similar to Example 2 | m/z 599.4 [M + H]⁺ | **** |
| Compound 22 | Similar to Example 2 | m/z 582.2 [M + H]⁺ | *** |
| Compound 23 | Similar to Example 2 | m/z 611.2 [M + H]⁺ | Less than |
| | | | zero |
| Compound 24 | Similar to Example 2 | m/z 568.4 [M + H]⁺ | *** |

¹See Example 6

### Example 3

### Compound 32: (R)-8-(imidazo[1,2-a]pyrazin-8-yl)-3-(2-(6-methyl-2-oxo-4-(1-phenylethoxy)pyridin-1(2H)-yl)ethyl)-1,3,8-triazaspiro[4.5]decane-2,4-dione

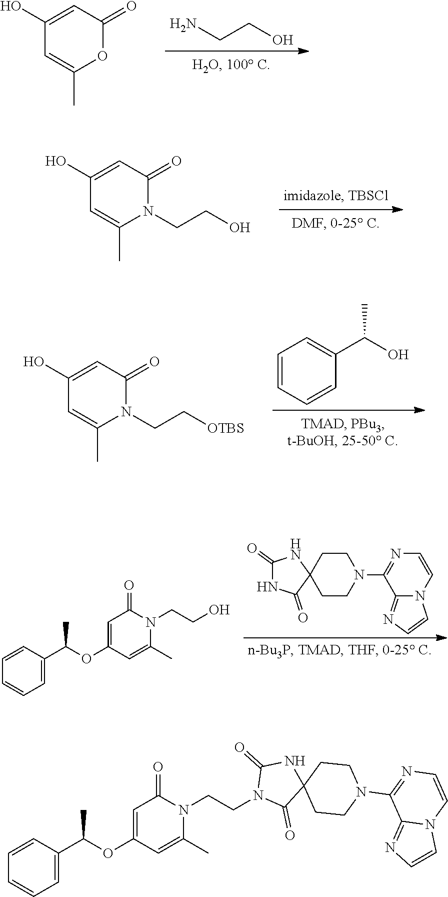

### Step 1: 4-hydroxy-1-(2-hydroxyethyl)-6-methylpyridin-2(1H)-one

A solution of 2-aminoethanol (4.84 g, 79.30 mmol, 1 equiv) and 4-hydroxy-6-methyl-pyran-2-one (10.00 g, 79.30 mmol, 1 equiv) in H₂O (100 mL) was stirred at 100° C. for 1 h. The reaction mixture was concentrated under the reduced pressure to give the title compound (13.00 g, crude) as a yellow solid.

LC-MS [ESI, M+1]: 169.8.

### Step 2: 1-(2-((tert-butyldimethylsilyl)oxy)ethyl)-4-hydroxy-6-methylpyridin-2(1H)-one

To a mixture of 4-hydroxy-1-(2-hydroxyethyl)-6-methyl-pyridin-2-one (13.00 g, 76.84 mmol, 1 equiv) and imidazole (5.23 g, 76.84 mmol, 1 equiv) in DMF (60 mL) was added TBSCl (12.74 g, 84.53 mmol, 1.1 equiv) at 0° C., then the mixture was stirred at 25° C. for 1 h. The reaction mixture was extracted with ethyl acetate (50 mL×3), the combined organic phase was washed with brine (30 mL), dried, filtered and concentrated in to afford the title compound (13.00 g, 59%) as a white solid.

¹H NMR (400 MHz, DMSO-d₆): δ 10.39 (s, 1H), 5.74 (d, J=2.0 Hz, 1H), 5.50 (d, J=2.0 Hz, 1H), 3.93 (t, J=5.6 Hz, 2H), 3.76 (t, J=5.2 Hz, 2H), 2.32 (s, 3H), 0.80 (s, 9H), 0.00 (s, 6H).

LC-MS [ESI, M+1]: 284.3.

### Step 3: (R)-1-(2-hydroxyethyl)-6-methyl-4-(1-phenylethoxy)pyridin-2(1H)-one

To a solution of TMAD (2.11 g, 12.28 mmol, 1.5 equiv) in t-BuOH (25 mL) was added tributylphosphane (2.48 g, 12.28 mmol, 1.5 equiv) dropwise at 25° C. The mixture was stirred at this temperature for 10 min, and then 1-(2-((tert-butyldimethylsilyl)oxy)ethyl)-4-hydroxy-6-methylpyridin-2(1H)-one (3.48 g, 12.28 mmol, 1.5 equiv) and (1S)-1-phenylethanol (1.00 g, 8.19 mmol, 1 equiv) in t-BuOH (25 mL) was added dropwise at 0° C. The mixture was stirred at 50° C. for 50 min. The reaction mixture was extracted with ethyl acetate (30 mL×3), the combined organic phase was washed with brine (30 mL), dried, filtered and concentrated to give a residue, which was purified by column chromatography (SiO₂, Petroleum ether:Ethyl acetate=3:1, R_{f}=0.5), and further purified by prep-HPLC (FA condition column: Phenomenex luna C18 150*25 mm*10 μm; mobile phase: (water(FA)-ACN); B %: 56%-86%, 10 min) to afford the title compound (200 mg, 9%) as a yellow oil.

¹H NMR (400 MHz, CDCl₃): δ 8.06 (s, 1H), 7.37-7.27 (m, 5H), 5.97 (s, 1H), 5.89 (s, 1H, 5.30 (q, J=6.4 Hz, 1H), 4.16 (s, 2H), 3.89 (s, 2H), 2.36 (s, 3H), 1.62 (d, J=6.4 Hz, 3H).

### Step 4: (R)-8-(imidazo[1,2-a]pyrazin-8-yl)-3-(2-(6-methyl-2-oxo-4-(1-phenylethoxy)pyridin-1(2H)-yl)ethyl)-1,3,8-triazaspiro[4.5]decane-2,4-dione

To a solution of TMAD (57 mg, 0.33 mmol, 1.5 equiv) in THF (1 mL) was added tributylphosphane (67 mg, 0.33 mmol, 1.5 equiv) dropwise at 0° C. After addition, the mixture was stirred at this temperature for 10 min, and then (R)-1-(2-hydroxyethyl)-6-methyl-4-(1-phenylethoxy)pyridin-2(1H)-one (60 mg, 0.22 mmol, 1 equiv) and 8-imidazo[1,2-a]pyrazin-8-yl-1,3,8-triazaspiro[4.5]decane-2,4-dione (94 mg, 0.33 mmol, 1.5 equiv) in THF (1 mL) was added dropwise at 0° C. The mixture was stirred at 25° C. for 50 min. The reaction mixture was extracted with ethyl acetate (10 mL×3), washed with brine (10 mL), the combined organic phase were dried, filtered and concentrated to give a residue, which was purified by prep-HPLC (column: Waters xbridge 150*25 mm 10 μm; mobile phase: (water(NH₄HCO₃)-ACN); B %: 22%-52%, 8 min) to afford the title compound (10.8 mg, 9%) as a white solid.

1H NMR (400 MHz, CDCl₃): δ 7.56 (d, J=0.8 Hz, 1H), 7.53 (d, J=4.4 Hz, 1H), 7.51 (d, J=1.2 Hz, 1H), 7.37 (d, J=4.4 Hz, 1H), 7.32-7.29 (m, 4H), 7.23-7.19 (m, 1H), 6.22 (s, 1H), 5.82 (d, J=2.0 Hz, 1H), 5.61 (d, J=2.8 Hz, 1H), 5.25 (q, J=6.4 Hz, 1H), 5.02-5.01 (m, 2H), 4.23-4.18 (m, 2H), 3.83-3.74 (m, 4H), 2.33 (s, 3H), 2.17-1.95 (m, 2H), 1.79-1.67 (m, 2H), 1.59 (d, J=6.4 Hz, 3H).

LC-MS [ESI, M+1]: 542.2.

DSF: ** (see Example 6)

### Example 4

### Compound 11: 3-(2-(6-methyl-4-(3-methyl-3-phenylpyrrolidin-1-yl)-2-oxopyridin-1(2H)-yl)ethyl)-7-(pyrimidin-2-yl)-1,3,7-triazaspiro[4.4]nonan-2-one

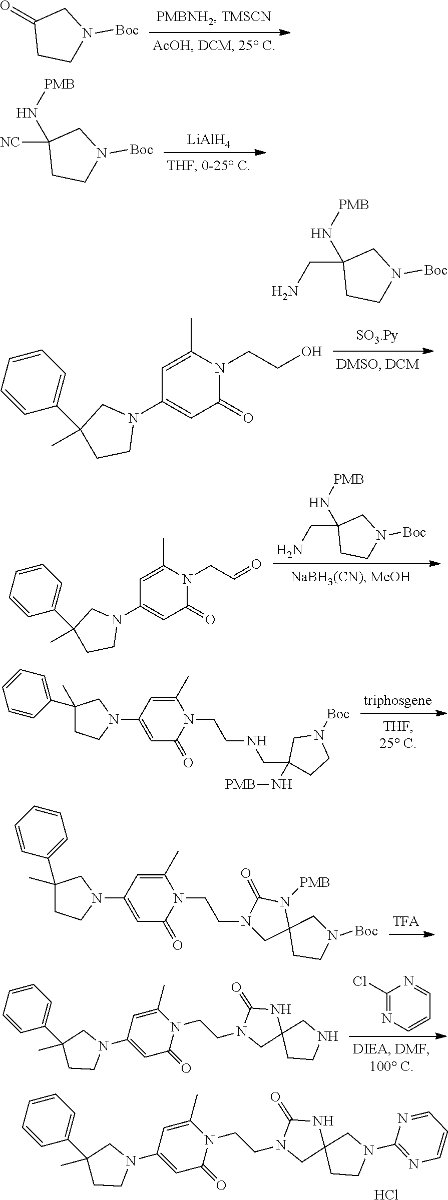

### Step 1: tert-butyl 3-cyano-3-((4-methoxybenzyl)amino)pyrrolidine-1-carboxylate

To a solution of tert-butyl 3-oxopyrrolidine-1-carboxylate (3 g, 16.20 mmol) in AcOH (25 mL) and DCM (25 mL) was added PMBNH₂ (2.67 g, 19.44 mmol) and TMSCN (4.02 g, 40.49 mmol) at 0° C. The reaction mixture was stirred for 16 h at 25° C. LCMS showed the starting material was consumed, the desired product mass wasn't detected. TLC showed the starting material was consumed and three new spots were observed. The mixture was quenched by H₂O (20 mL), The resulting mixture was transferred to a separatory funnel and the aqueous layer mixture was extracted with DCM (30 mL×3). The combined organic layers were washed with brine (20 mL), dried over anhydrous sodium sulfate, filtered, and concentrated under reduced pressure. The residue was purified by flash silica gel chromatography (20 g Silica Flash Column, Eluent of 0-50% Ethyl acetate/Petroleum ether gradient at 80 mL/min) to afford the title compound (2.2 g, 41% yield) as yellow gum.

¹H NMR (400 MHz, CDCl₃) δ 7.30-7.24 (m, 2H), 6.87 (bd, J=8.4 Hz, 2H), 4.12 (q, J=7.2 Hz, 1H), 3.97-3.67 (m, 6H), 3.66-3.39 (m, 3H), 2.39-2.27 (m, 1H), 2.15 (td, J=6.4, 12.8 Hz, 1H), 1.49-1.43 (m, 9H)

### Step 2: tert-butyl 3-(aminomethyl)-3-((4-methoxybenzyl)amino)pyrrolidine-1-carboxylate

To a solution of tert-butyl 3-cyano-3-((4-methoxybenzyl)amino)pyrrolidine-1-carboxylate (1 g, 3.02 mmol) in THF (20 mL) was added LiAlH₄ (172 mg, 4.53 mmol) under N₂ at 0° C. The mixture was stirred for 2 h at 25° C. LCMS showed the starting material was consumed, the desired product mass was detected. The mixture was quenched by Na₂SO₄·10H₂O (1.75 g). The suspension was filtered and the filter cake was washed with EtOAc (30 mL). The filtrate was concentrated under reduced pressure. The title compound (800 mg, 79% yield) as yellow gum was used into next step without purification.

LC-MS [ESI, M+1]: 336.3

¹H NMR (400 MHz, CDCl₃) δ 7.25-7.16 (m, 2H), 6.88-6.84 (m, 2H), 3.79 (s, 3H), 3.74-3.21 (m, 8H), 1.45 (s, 9H), 1.43 (s, 2H).

### Step 3: 2-(6-methyl-4-(3-methyl-3-phenylpyrrolidin-1-yl)-2-oxopyridin-1(2H)-yl)acetaldehyde

To a solution of 1-(2-hydroxyethyl)-6-methyl-4-(3-methyl-3-phenyl-pyrrolidin-1-yl)pyridin-2-one (500 mg, 1.60 mmol) and DIEA (621 mg, 4.80 mmol) in DCM (3 mL) and DMSO (3 mL) was added SO₃·Py (764 mg, 4.80 mmol) under N₂ at 0° C. The mixture was stirred for 16 h at 25° C. LCMS showed the starting material was consumed, the desired product mass was detected. The mixture was quenched with water (20 mL), then extracted with EtOAc (30 mL×3). The organic was washed with brine (50 mL), dried over Na₂SO₄, filtered and concentrated under reduced pressure. The residue was purified by flash silica gel chromatography (25 g Silica Flash Column, Eluent of 50˜100% Ethyl acetate/Petroleum ether gradient at 60 mL/min). to afford the title compound (240 mg, 48% yield) as colorless oil.

LC-MS [ESI, M+1]: 311.2

### Step 4: tert-butyl 3-((4-methoxybenzyl)amino)-3-(((2-(6-methyl-4-(3-methyl-3-phenylpyrrolidin-1-yl)-2-oxopyridin-1(2H)-yl)ethyl)amino)methyl)pyrrolidine-1-carboxylate

To a solution of 2-[2-methyl-4-(3-methyl-3-phenyl-pyrrolidin-1-yl)-6-oxo-1-pyridyl]acetaldehyde (220 mg, 0.71 mmol) and tert-butyl 3-(aminomethyl)-3-[(4-methoxyphenyl)methylamino]pyrrolidine-1-carboxylate (285 mg, 0.85 mmol) in MeOH (4 mL) was added AcOH (0.71 mmol, 0.04 mL) at 25° C. The mixture was stirred for 1 h and then NaBH₃CN (134 mg, 2.13 mmol) was added to the mixture. The reaction was stirred for 16 h at 25° C. LCMS showed the starting material was consumed, the desired product mass was detected. The mixture was quenched by saturated sodium bicarbonate (5 mL). The resulting mixture was transferred to a separatory funnel, and the aqueous layer mixture was extracted with ethyl acetate (15 mL×3). The combined organic layers were washed with brine (10 mL), dried over anhydrous sodium sulfate, filtered, and concentrated under reduced pressure. The residue was purified by flash silica gel chromatography (12 g Silica Flash Column, Eluent of 0˜10% Methanol/Dichloromethane@ 40 mL/min) to afford the title compound (160 mg, 36% yield) as yellow gum.

LC-MS [ESI, M+1]: 630.2

### Step 5: tert-butyl 1-(4-methoxybenzyl)-3-(2-(6-methyl-4-(3-methyl-3-phenylpyrrolidin-1-yl)-2-oxopyridin-1(2H)-yl)ethyl)-2-oxo-1,3,7-triazaspiro[4.4]nonane-7-carboxylate

A solution of tert-butyl 3-[(4-methoxyphenyl)methylamino]-3-[[2-[2-methyl-4-(3-methyl-3-phenyl-pyrrolidin-1-yl)-6-oxo-1-pyridyl]ethylamino]methyl]pyrrolidine-1-carboxylate (140 mg, 0.22 mmol) in THF (5 mL) was added triphosgene (26 mg, 0.09 mol) at 25° C. The mixture was stirred for 16 h at 25° C. LCMS showed the starting material remained, the desired product mass was detected. The mixture was concentrated under reduced pressure. The residue was purified by flash silica gel chromatography (12 g Silica Flash Column, Eluent of 0-10% Methanol/Dichloromethane at 40 mL/min) to afford the title compound (50 mg, 34% yield) as colorless gum.

LC-MS [ESI, M+1]: 656.3

### Step 6: 3-(2-(6-methyl-4-(3-methyl-3-phenylpyrrolidin-1-yl)-2-oxopyridin-1(2H)-yl)ethyl)-1,3,7-triazaspiro[4.4]nonan-2-one

A solution of tert-butyl 1-[(4-methoxyphenyl)methyl]-3-[2-[2-methyl-4-(3-methyl-3-phenyl-pyrrolidin-1-yl)-6-oxo-1-pyridyl]ethyl]-2-oxo-1,3,7-triazaspiro[4.4]nonane-7-carboxylate (40 mg, 0.06 mmol) in TFA (0.5 mL) was stirred for 16 h at 60° C. LCMS showed the starting material was consumed, the desired product mass was detected. The mixture was concentrated under reduced pressure. The title compound (60 mg, crude, TFA) as pink gum was used into next step without purification.

LC-MS [ESI, M+1]: 436.3

### Step 7: 3-(2-(6-methyl-4-(3-methyl-3-phenylpyrrolidin-1-yl)-2-oxopyridin-1(2H)-yl)ethyl)-7-(pyrimidin-2-yl)-1,3,7-triazaspiro[4.4]nonan-2-one

To a solution of 3-[2-[2-methyl-4-(3-methyl-3-phenyl-pyrrolidin-1-yl)-6-oxo-1-pyridyl]ethyl]-1,3,7-triazaspiro[4.4]nonan-2-one (60 mg, 0.11 mmol, TFA) in DMF (1 mL) was added DIEA (42 mg, 0.33 mmol) and 2-chloropyrimidine (15 mg, 0.13 mol). The mixture was stirred for 16 h at 100° C. LCMS showed the starting material was consumed, the desired product mass was detected. The mixture was concentrated under reduced pressure. The residue was purified by prep-HPLC (column: Welch Xtimate C18 150×25 mm×5 μm; mobile phase: [water(HCl)-ACN]; B %: 16%-46%, 8 min) to afford the title compound (11.71 mg, 19% yield, 95.2% purity, HCl) as yellow solid.

LC-MS [ESI, M+1]: 514.4

¹H NMR (400 MHz, METHANOL-d₄) δ=8.68-8.64 (m, 2H), 7.36 (d, J=3.6 Hz, 4H), 7.28-7.23 (m, 1H), 7.06-7.04 (m, 1H), 6.62-6.51 (m, 1H), 4.93-4.91 (m, 1H), 4.34 (t, J=6.0 Hz, 2H), 3.86-3.81 (m, 4H), 3.74-3.48 (m, 8H), 2.62 (d, J=4.4 Hz, 3H), 2.43-2.29 (m, 4H), 1.42 (d, J=5.2 Hz, 3H).

DSF: *** (see Example 6)

| Compound Number | Synthesis | LC/MS | DSF¹ |
|---|---|---|---|
| Compound 12 | Similar to Example 4 | m/z 567.4 [M + H]⁺ | *** |
| Compound 13 | Similar to Example 4 | m/z 581.3 [M + H]⁺ | ** |
| Compound 33 | Similar to Example 4 | N.D. | *** |

¹See Example 6

### Example 5

### Compound 34: 3-(2-(6-methyl-4-(3-methyl-2-oxo-3-phenylpyrrolidin-1-yl)-2-oxopyridin-1(2H)-yl)ethyl)-7-(pyrimidin-2-yl)-1,3,7-triazaspiro[4.4]nonane-2,4-dione

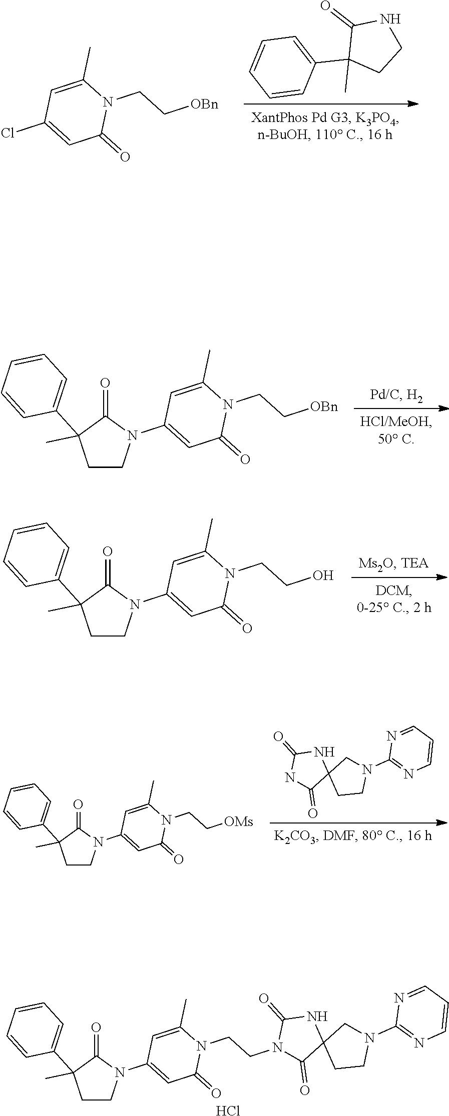

### Step 1: 1-(2-(benzyloxy)ethyl)-6-methyl-4-(3-methyl-2-oxo-3-phenylpyrrolidin-1-yl)pyridin-2(1H)-one

To a solution of 1-(2-benzyloxyethyl)-4-chloro-6-methyl-pyridin-2-one (500 mg, 1.80 mmol) in n-BuOH (5 mL) was added 3-methyl-3-phenyl-pyrrolidin-2-one (315 mg, 1.80 mmol) and K₃PO₄ (1.15 g, 5.40 mmol) and XantPhos Pd G3 (171 mg, 0.18 mmol). The mixture was stirred at 110° C. for 16 h under N₂. LCMS showed the starting material was consumed completely and the desired mass was detected. The mixture was filtered, and concentrated under reduced pressure affording the residue. The residue was purified by column chromatography (SiO₂, Petroleum ether:Ethyl acetate=100:1 to 1:10) to give the title compound (500 mg, 67% yield) as a yellow oil.

LC-MS [ESI, M+1]: 417.4

### Step 2: 1-(2-hydroxyethyl)-6-methyl-4-(3-methyl-2-oxo-3-phenylpyrrolidin-1-yl)pyridin-2(1H)-one

To a solution of 1-(2-benzyloxyethyl)-6-methyl-4-(3-methyl-2-oxo-3-phenyl-pyrrolidin-1-yl)pyridin-2-one (500 mg, 1.20 mmol) in MeOH (10 mL) was added Pd/C (50 mg, 10% purity) and HCl (12 M, 0.5 mL) under N₂. The suspension was degassed under vacuum and purged with H₂ three times. The mixture was stirred under H₂ (50 psi) at 50° C. for 16 h. LCMS showed the starting material was consumed completely and the desired mass was detected. The suspension was filtered and the filtered cake was washed with MeOH (20 mL). The filtrate was concentrated under reduced pressure. The residue was purified by column chromatography (SiO₂, Petroleum ether:Ethyl acetate=100:1 to 0:1) to afford the title compound (200 mg, 46% yield) as a white solid. Then 50 mg of this compound was purified again by prep-HPLC (HCl condition; column: Welch Ultimate AQ-C18 150×30 mm×5 μm; mobile phase: [water(HCl)-ACN]; B %: 21%-51%, 10 min) to afford the title compound (32.03 mg, 8% yield, 99.8% purity) as a white solid.

LC-MS [ESI, M+1]: 327.3

¹H NMR (400 MHz, METHANOL-d₄) δ=7.56 (d, J=2.2 Hz, 1H), 7.43-7.38 (m, 2H), 7.35 (t, J=7.6 Hz, 2H), 7.30-7.23 (m, 2H), 4.38 (t, J=5.4 Hz, 2H), 3.98-3.87 (m, 3H), 3.77-3.68 (m, 1H), 2.73-2.66 (m, 4H), 2.34 (d, J=13.2 Hz, 1H), 1.59 (s, 3H).

### Step 3: 2-(6-methyl-4-(3-methyl-2-oxo-3-phenylpyrrolidin-1-yl)-2-oxopyridin-1(2H)-yl)ethyl methanesulfonate

To the solution of 1-(2-hydroxyethyl)-6-methyl-4-(3-methyl-2-oxo-3-phenylpyrrolidin-1-yl)pyridin-2(1H)-one (80 mg, 0.25 mmol) and DIEA (63 mg, 0.49 mmol) in DCM (1 mL) was added Ms₂O (51 mg, 0.29 mmol) at 0° C., then the mixture was stirred at 25° C. for 2 h. LCMS showed the desired mass was detected, the starting material was consumed. The mixture was concentrated under reduced pressure to afford the title compound (90 mg, 90.8% yield) as a yellow solid.

### Step 4: 3-(2-(6-methyl-4-(3-methyl-2-oxo-3-phenylpyrrolidin-1-yl)-2-oxopyridin-1(2H)-yl)ethyl)-7-(pyrimidin-2-yl)-1,3,7-triazaspiro[4.4]nonane-2,4-dione hydrochloride

To the mixture of 7-pyrimidin-2-yl-1,3,7-triazaspiro[4.4]nonane-2,4-dione (52 mg, 0.22 mmol) and K₂CO₃ (62 mg, 0.45 mmol) in DMF (1 mL) was added 2-(6-methyl-4-(3-methyl-2-oxo-3-phenylpyrrolidin-1-yl)-2-oxopyridin-1(2H)-yl)ethyl methanesulfonate (90 mg, 0.22 mmol), then the mixture was stirred at 80° C. for 16 h. LCMS showed the desired mass was detected, the starting material was consumed. The mixture was filtered and the filtrate was purified by prep-HPLC (column: Phenomenex C18 150×25 mm×10 μm; mobile phase: [water(NH₄HCO₃)-ACN]; B %: 20%-50%, 8 min), then purified by prep-HPLC (column: Phenomenex luna C18 150×25 mm×10 μm; mobile phase: [water(HCl)-ACN]; B %: 19%-49%, 10 min) to afford the title compound (27.16 mg, 21% yield, 97.6% purity, HCl salt) as a white solid.

LC-MS [ESI, M+1]: 542.4

¹H NMR (400 MHz, CDCl₃) δ=8.93 (s, 1H), 8.58 (s, 2H), 8.10 (s, 1H), 7.52-7.26 (m, 5H), 7.23 (s, 1H), 6.98 (s, 1H), 4.55-3.76 (m, 10H), 2.70-2.27 (m, 7H), 1.56 (s, 3H).

DSF: * (see Example 6)

### Example 6

Differential Scanning Fluorimetry Assay

The stabilization effects the compounds have on the immunoglobulin light chain protein was quantitively measured using a thermal shift assay. Recombinant amyloidogenic full length light chain protein WIL-FL T46L was produced in E. coli, purified, and used in the assay. The Thermal Shift Protein Stability Kit from Biotium was used and the assay was done in 96-well Plates following the manufacturer's instructions. The protein and compounds were incubated at RT for 30 min before adding the dye and subjected to the thermal shift assay. The thermal stability (Tm) of each sample was then determined using standard curve fitting methods and compared to vehicle. Data are expressed as temperature shift: [Tm]_{compound}−[Tm]_{vehicle}.

Results

The compounds provided herein induce shifts in the ranges below:
0-0.5° C.═*
>0.5-1° C.═**
>1-1.5° C.═***
>1.5-2.5° C.═****
>2.5° C.═*****

The results for each compound provided herein are shown in Examples 1-5, above.

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound of Formula I: 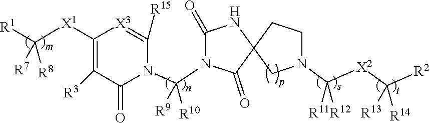
or a pharmaceutically acceptable derivative thereof, wherein
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl, heteroaryl or heterocycloalkyl;
R² is aryl or heteroaryl;
R³ to R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

2. The compound of claim 1 that has Formula Ia: 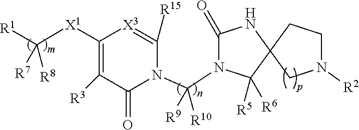
or a pharmaceutically acceptable derivative thereof, wherein
n is an integer from 1-4;
p is an integer from 1-3;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

3. The compound of claim 1 that has Formula Ib: 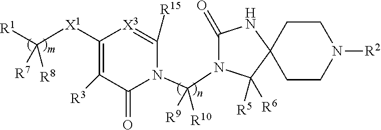
or a pharmaceutically acceptable derivative thereof, wherein
n is an integer from 1-4;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

4. The compound of claim 1 that has Formula Ic: 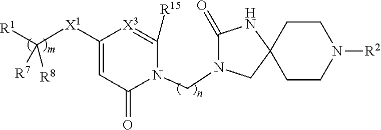
or a pharmaceutically acceptable derivative thereof, wherein
n is an integer from 1-4;
m is an integer from 0-3;
X¹ is a bond, O, NR¹⁶ or CO;
X³ is N or CH;
R¹ is aryl, heteroaryl or heterocycloalkyl;
R² is heteroaryl;
R⁷ and R⁸ are each independently H, alkyl or aralkyl, or together with the carbon atom to which they are attached form cycloalkyl; and
R¹⁵ and R¹⁶ are each independently H or alkyl.

5. The compound of claim 1, wherein R¹ is aryl or heterocycloalkyl.

6. The compound of claim 1, wherein R¹ is aryl.

7. The compound of claim 1, wherein R¹ is heterocycloalkyl.

8. The compound of claim 1 that has Formula Id: 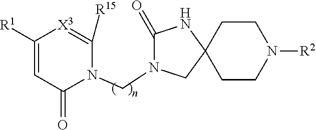
or a pharmaceutically acceptable derivative thereof, wherein
n is 2 or 3;
X³ is N or CH;
R¹ is heterocycloalkyl, optionally substituted with 1-3 substituents each independently selected from alkyl and aryl;
R² is heteroaryl; and
R¹⁵ is alkyl.

9. The compound of claim 1 that has Formula Ie: 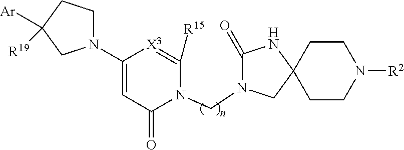
or a pharmaceutically acceptable derivative thereof, wherein
n is 2 or 3;
X³ is N or CH;
Ar is aryl;
R² is heteroaryl;
R¹⁵ is alkyl; and
R¹⁹ is alkyl.

10. The compound of claim 9, wherein R¹⁹ is lower alkyl.

11. The compound of claim 9, wherein R¹⁹ is methyl or ethyl.

12. The compound of claim 9, wherein R¹⁹ is methyl.

13. The compound of claim 9, wherein R¹⁹ is ethyl.

14. The compound of claim 1 that has the formula:

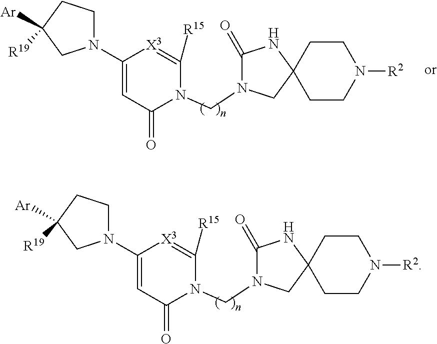

15. The compound of claim 1 that has Formula If: 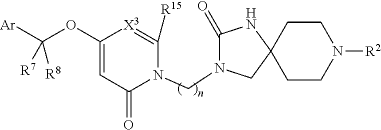
or a pharmaceutically acceptable derivative thereof, wherein
n is 2 or 3;
X³ is N or CH;
Ar is aryl;
R² is heteroaryl;
R¹⁵ is alkyl; and
R⁷ and R⁸ are each independently H or alkyl, or together with the carbon atom to which they are attached form cycloalkyl.

16. The compound of claim 1, wherein R⁷ is alkyl and R⁸ is H.

17. The compound of claim 1, wherein R⁷ is methyl and R⁸ is H.

18. The compound of claim 1, wherein R⁷ and R⁸ together with the carbon atom to which they are attached form cyclopropyl.

19. The compound of claim 1 that has the formula:

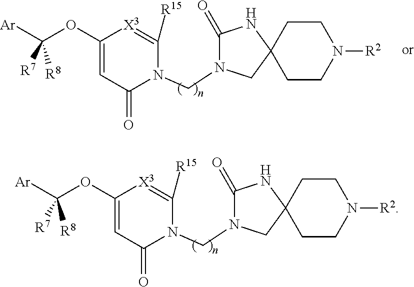

20. A compound of claim 1 that has Formula Ig: 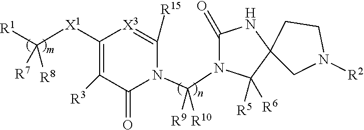
or a pharmaceutically acceptable derivative thereof, wherein
n is an integer from 1-4;
m is an integer from 0-3;
X¹ is a bond, O, NR¹⁶ or CO;
X³ is N or CR⁴;
R¹ is aryl, heteroaryl or heterocycloalkyl;
R² is heteroaryl;
R³ to R⁶, R⁹, R¹⁰, R¹⁵ and R¹⁶ are each independently H, alkyl or aralkyl; and
R⁷ and R⁸ are each independently H, alkyl or aralkyl, or together with the carbon atom to which they are attached form cycloalkyl.

21. The compound of claim 1 that has Formula Ih: 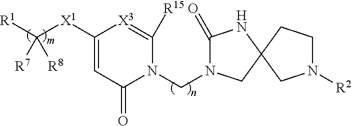
or a pharmaceutically acceptable derivative thereof, wherein
n is an integer from 1-4;
m is an integer from 0-3;
X¹ is a bond, O, NR¹⁶ or CO;
X³ is N or CH;
R¹ is aryl, heteroaryl or heterocycloalkyl;
R² is heteroaryl;
R⁷ and R⁸ are each independently H, alkyl or aralkyl, or together with the carbon atom to which they are attached form cycloalkyl; and
R¹⁵ and R¹⁶ are each independently H or alkyl.

22. The compound of claim 20, wherein R¹ is aryl or heterocycloalkyl.

23. The compound of claim 20, wherein R¹ is aryl.

24. The compound of claim 20, wherein R¹ is heterocycloalkyl.

25. The compound of claim 1 that has Formula Ii: 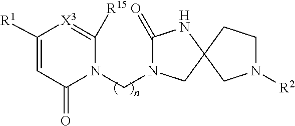
or a pharmaceutically acceptable derivative thereof, wherein
n is 2 or 3;
X³ is N or CH;
R¹ is heterocycloalkyl, optionally substituted with 1-3 substituents each independently selected from alkyl and aryl;
R² is heteroaryl; and
R¹⁵ is alkyl.

26. The compound of claim 1 that has Formula Ij: 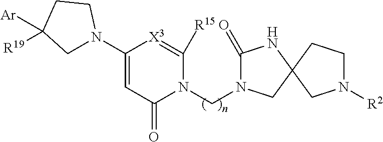
or a pharmaceutically acceptable derivative thereof, wherein
n is 2 or 3;
X³ is N or CH;
Ar is aryl;
R² is heteroaryl;
R¹⁵ is alkyl; and
R¹⁹ is alkyl.

27. The compound of claim 1, wherein R¹⁹ is lower alkyl.

28. The compound of claim 1, wherein R¹⁹ is methyl or ethyl.

29. The compound of claim 1 that has the formula:

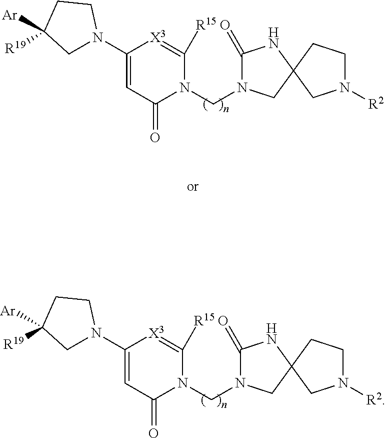

30. The compound of claim 1 that has Formula Ik: 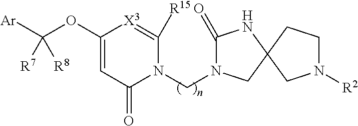
or a pharmaceutically acceptable derivative thereof, wherein
n is 2 or 3;
X³ is N or CH;
Ar is aryl;
R² is heteroaryl;
R¹⁵ is alkyl; and
R⁷ and R⁸ are each independently H or alkyl, or together with the carbon atom to which they are attached form cycloalkyl.

31. The compound of claim 1, wherein R⁷ is alkyl and R⁸ is H.

32. The compound of claim 1, wherein R⁷ is methyl and R⁸ is H.

33. The compound of claim 1, wherein R⁷ and R⁸ together with the carbon atom to which they are attached form cyclopropyl.

34. The compound of claim 1, that has the formula:

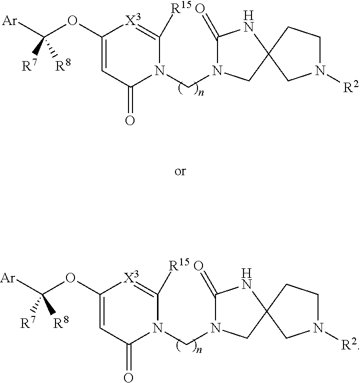

35. The compound of claim 1, wherein n is 2.

36. The compound of claim 1, wherein n is 3.

37. The compound of claim 1, wherein X³ is N.

38. The compound of claim 1, wherein X³ is CH.

39. The compound of claim 1, wherein Ar is unsubstituted aryl.

40. The compound of claim 1, wherein Ar is optionally substituted phenyl.

41. The compound of claim 1, wherein Ar is unsubstituted phenyl.

42. The compound of claim 1, wherein R² is bicyclic or monocyclic heteroaryl.

43. The compound of claim 1, wherein R² is triazolopyrazinyl, imidazopyrazinyl, pyrazolopyrimidinyl, pyridyl, pyridazinyl or pyrimidinyl.

44. The compound of claim 1, wherein R² is triazolopyrazinyl, imidazopyrazinyl, pyrazolopyrimidinyl or pyrimidinyl.

45. The compound of claim 1, wherein R² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl, 2-pyridyl, 3-pyridyl, 3-pyridazinly or 2-pyrimidinyl.

46. The compound of claim 1, wherein R² is [1,2,4]-triazolo[4,3-a]pyrazin-8-yl, imidazo[1,5-a]pyrazin-8-yl, 5-carboxyimidazo[1,5-a]pyrazin-8-yl, imidazo[1,2-a]pyrazin-8-yl, 1H-pyrazolo[3,4-d]pyrimidin-4-yl, 6-carboxy-3-pyridyl, 6-carboxy-2-pyridyl, 6-ethoxycarbonyl-2-pyridyl, 6-ethoxycarbonyl-3-pyridyl, 6-carboxy-3-pyridazinyl, 6-ethoxycarbonyl-3-pyridazinyl or 2-pyrimidinyl.

47. The compound of claim 1, wherein R² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl or 2-pyrimidinyl.

48. The compound of claim 1, wherein R¹⁵ is lower alkyl.

49. The compound of claim 1, wherein R¹⁵ is methyl.

50. The compound of claim 1 that has Formula Im: 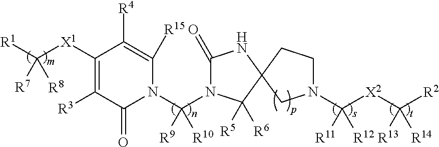
or a pharmaceutically acceptable derivative thereof, wherein
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl or heterocycloalkyl;
R² is aryl or heteroaryl; and
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

51. The compound of claim 1, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

52. The compound of claim 1, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁸ to R¹⁴ are each independently H; and
R⁷ and R¹⁵ are each independently alkyl.

53. The compound of claim 1, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

54. The compound of claim 1, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³, R⁵, R⁶ and R⁸ to R¹⁴ are each independently H; and
R⁷ and R¹⁵ are each independently alkyl.

55. The compound of claim 1 that has Formula In: 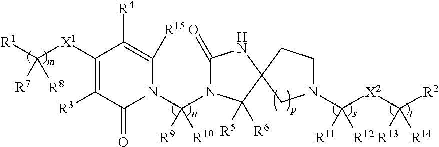
or a pharmaceutically acceptable derivative thereof, wherein
n is an integer from 1-4;
p is an integer from 1-3;
m, s and t are each independently an integer from 0-3;
X¹ is a bond, O, NR¹⁶ or CO;
X² is a bond, CONR¹⁷, SO₂NR¹⁸, CO or SO₂;
X³ is N or CR⁴;
R¹ is aryl or heterocycloalkyl;
R² is aryl or heteroaryl;
R³ to R⁶ and R⁹ to R¹⁸ are each independently H, alkyl or aralkyl; and
R⁷ and R⁸ are each independently H, alkyl or aralkyl, or together with the carbon atom to which they are attached form cycloalkyl.

56. The compound of claim 1, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶ and R⁹ to R¹⁵ are each independently H, alkyl or aralkyl; and
R⁷ and R⁸ are each independently H, alkyl or aralkyl, or together with the carbon atom to which they are attached form cycloalkyl.

57. The compound of claim 1, or a pharmaceutically acceptable derivative thereof, wherein:
n is 2 or 3;
p is 2;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl; and
R³ to R⁵, R⁶ and R⁸ to R¹⁴ are each independently H; and
R⁷ and R¹⁵ are each independently alkyl.

58. The compound of claim 1, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁶ and R⁹ to R¹⁵ are each independently H, alkyl or aralkyl; and
R⁷ and RB are each independently H, alkyl or aralkyl, or together with the carbon atom to which they are attached form cycloalkyl.

59. The compound of claim 1, or a pharmaceutically acceptable derivative thereof, wherein:
n is 2 or 3;
p is 1;
m is 0 or 1;
s and t are each 0;
X¹ is a bond or O;
X² is a bond;
R¹ is aryl or heterocycloalkyl, each optionally substituted with 1-3 substituents each independently selected from alkyl and phenyl;
R² is heteroaryl;
R³ to R⁵, R⁶ and R to R¹⁴ are each independently H; and
R⁷ and R¹⁵ are each independently alkyl.

60. The compound of claim 55 that has Formula Io: 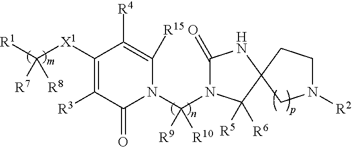
or a pharmaceutically acceptable derivative thereof, wherein:
n is an integer from 1-4;
p is an integer from 1-3;
m is an integer from 0-3;
X¹ is a bond, O, NR¹⁶ or CO;
R¹ is aryl or heterocycloalkyl;
R² is heteroaryl; and
R³ to R¹⁰, R¹⁵ and R¹⁶ are each independently H or alkyl.

61. The compound of claim 50 that has Formula Ij: 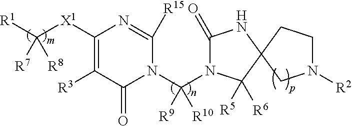
or a pharmaceutically acceptable derivative thereof, wherein:
n is an integer from 1-4;
p is an integer from 1-3;
m is an integer from 0-3;
X¹ is a bond, O, NR¹⁶ or CO;
R¹ is aryl or heterocycloalkyl;
R² is heteroaryl; and
R³, R⁵ to R¹⁰, R¹⁵ and R¹⁶ are each independently H or alkyl.

62. The compound of claim 50, wherein:
n is 2 or 3;
p is 2;
m is 0 or 1;
X¹ is a bond or O;
R¹ is aryl or heterocycloalkyl, each optionally substituted with 1-3 substituents each independently selected from alkyl and aryl;
R² is heteroaryl; and
R³, R⁵ to R¹⁰, R¹⁵ and R¹⁶ are each independently H or alkyl.

63. The compound of claim 50, wherein:
n is 2 or 3;
p is 1;
m is 0 or 1;
X¹ is a bond or O;
R¹ is aryl or heterocycloalkyl, each optionally substituted with 1-3 substituents each independently selected from alkyl and aryl;
R² is heteroaryl; and
R³, R⁵ to R¹⁰, R¹¹ and R¹⁶ are each independently H or alkyl.

64. The compound of claim 1 that is:

| Compound | Structure |
|---|---|
| 1 | 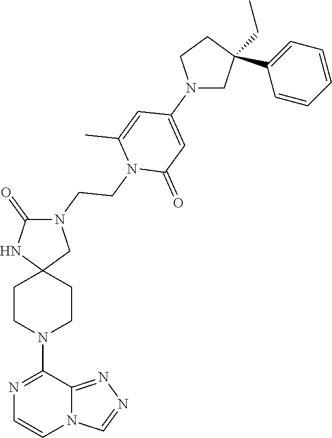 [SMILES: CC[C@@]1(c2ccccc2)CCN(c2cc(C)n(CCN3CC4(CCN(c5nccn6cnnc56)CC4)NC3=O)c(=O)c2)C1] |
| 2 | 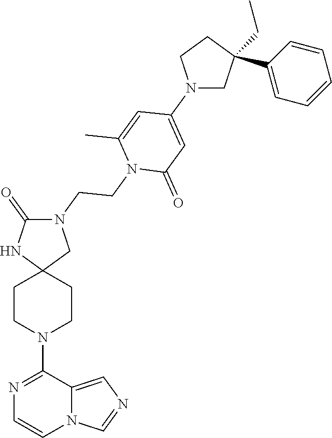 [SMILES: CC[C@@]1(c2ccccc2)CCN(c2cc(C)n(CCN3CC4(CCN(c5nccn6cncc56)CC4)NC3=O)c(=O)c2)C1] |
| 3 | 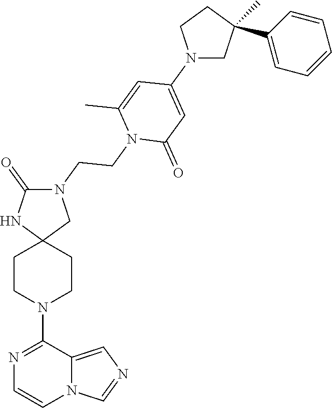 [SMILES: Cc1cc(N2CC[C@@](C)(c3ccccc3)C2)cc(=O)n1CCN1CC2(CCN(c3nccn4cncc34)CC2)NC1=O] |
| 4 | 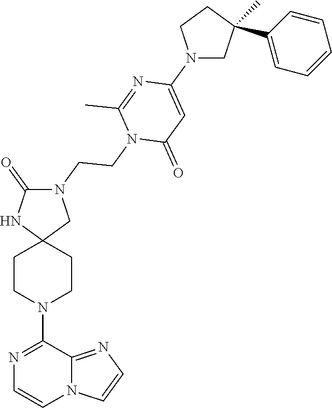 [SMILES: Cc1nc(N2CC[C@@](C)(c3ccccc3)C2)cc(=O)n1CCN1CC2(CCN(c3nccn4ccnc34)CC2)NC1=O] |
| 5 | 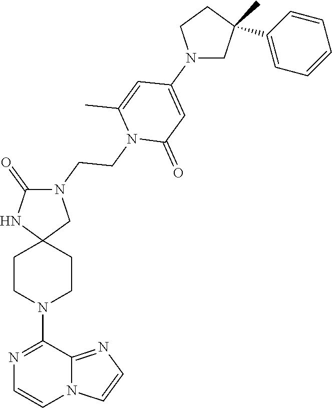 [SMILES: Cc1cc(N2CC[C@](C)(c3ccccc3)C2)cc(=O)n1CCN1CC2(CCN(c3nccn4ccnc34)CC2)NC1=O] |
| 6 | 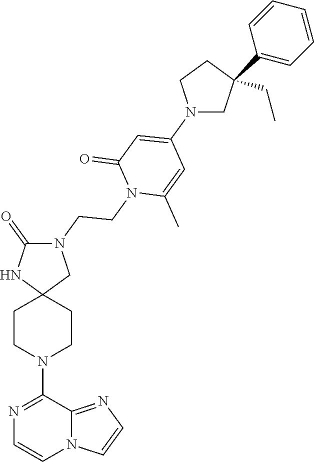 [SMILES: CC[C@@]1(c2ccccc2)CCN(c2cc(C)n(CCN3CC4(CCN(c5nccn6ccnc56)CC4)NC3=O)c(=O)c2)C1] |
| 7 | 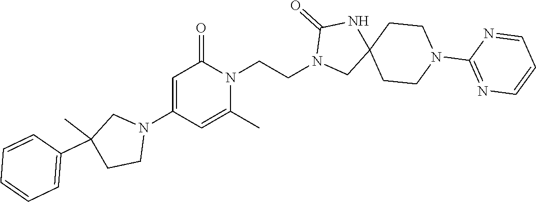 [SMILES: Cc1cc(N2CCC(C)(c3ccccc3)C2)cc(=O)n1CCN1CC2(CCN(c3ncccn3)CC2)NC1=O] |
| 8 | 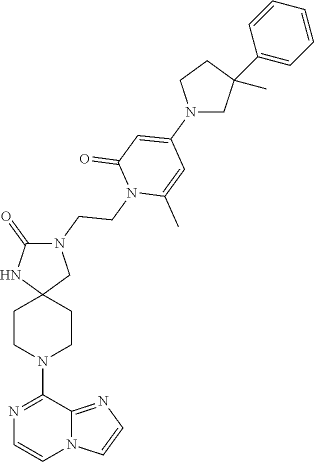 [SMILES: Cc1cc(N2CCC(C)(c3ccccc3)C2)cc(=O)n1CCN1CC2(CCN(c3nccn4ccnc34)CC2)NC1=O] |
| 9 | 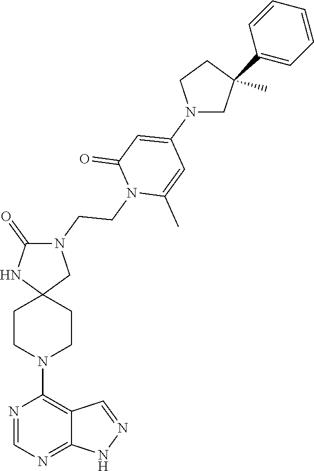 [SMILES: Cc1cc(N2CC[C@@](C)(c3ccccc3)C2)cc(=O)n1CCN1CC2(CCN(c3ncnc4[nH]ncc34)CC2)NC1=O] |
| 10 | 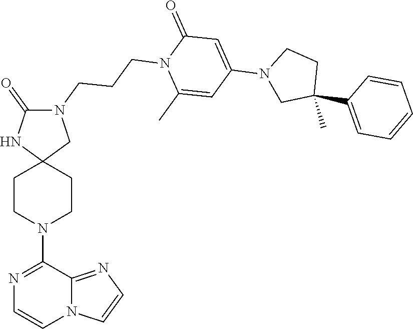 [SMILES: Cc1cc(N2CC[C@@](C)(c3ccccc3)C2)cc(=O)n1CCCN1CC2(CCN(c3nccn4ccnc34)CC2)NC1=O] |
| 11 | 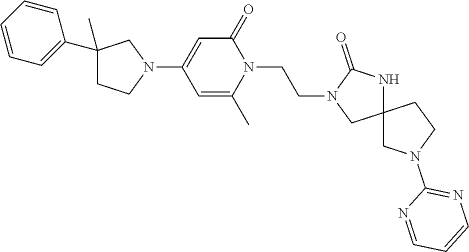 [SMILES: Cc1cc(N2CCC(C)(c3ccccc3)C2)cc(=O)n1CCN1CC2(CCN(c3ncccn3)C2)NC1=O] |
| 12 | 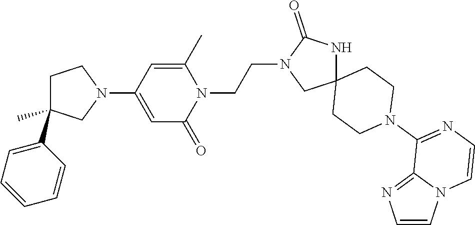 [SMILES: Cc1cc(N2CC[C@](C)(c3ccccc3)C2)cc(=O)n1CCN1CC2(CCN(c3nccn4ccnc34)CC2)NC1=O] |
| 13 | 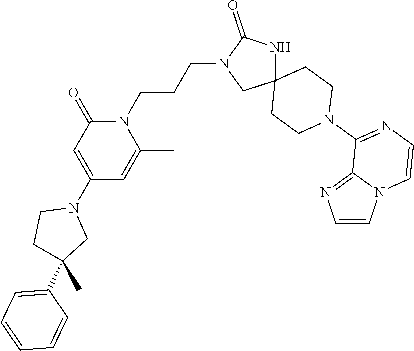 [SMILES: Cc1cc(N2CC[C@@](C)(c3ccccc3)C2)cc(=O)n1CCCN1CC2(CCN(c3nccn4ccnc34)CC2)NC1=O] |
| 14 | 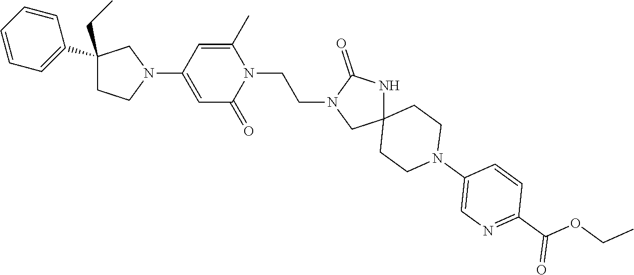 [SMILES: CCOC(=O)c1ccc(N2CCC3(CC2)CN(CCn2c(C)cc(N4CC[C@](CC)(c5ccccc5)C4)cc2=O)C(=O)N3)cn1] |
| 15 | 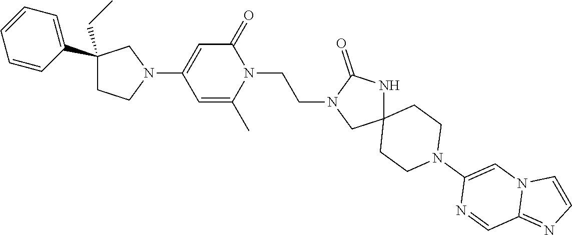 [SMILES: CC[C@@]1(c2ccccc2)CCN(c2cc(C)n(CCN3CC4(CCN(c5cn6ccnc6cn5)CC4)NC3=O)c(=O)c2)C1] |
| 16 | 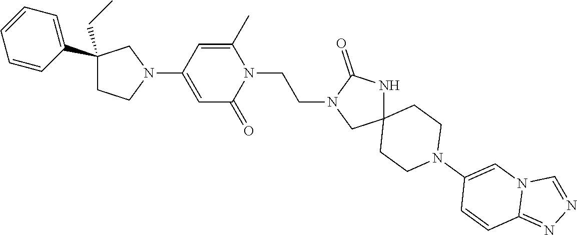 [SMILES: CC[C@@]1(c2ccccc2)CCN(c2cc(C)n(CCN3CC4(CCN(c5ccc6nncn6c5)CC4)NC3=O)c(=O)c2)C1] |
| 17 | 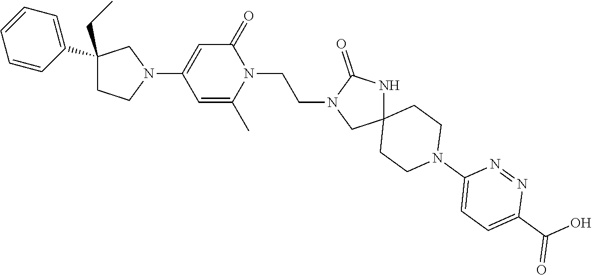 [SMILES: CC[C@]1(c2ccccc2)CCN(c2cc(C)n(CCN3CC4(CCN(c5ccc(C(=O)O)nn5)CC4)NC3=O)c(=O)c2)C1] |
| 18 | 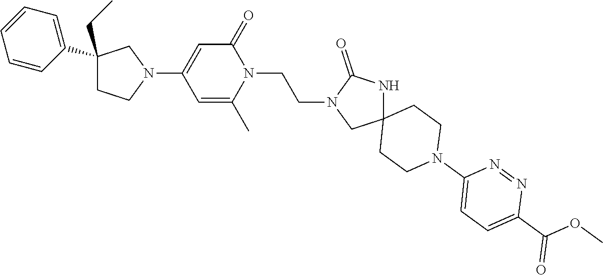 [SMILES: CC[C@]1(c2ccccc2)CCN(c2cc(C)n(CCN3CC4(CCN(c5ccc(C(=O)OC)nn5)CC4)NC3=O)c(=O)c2)C1] |
| 19 | 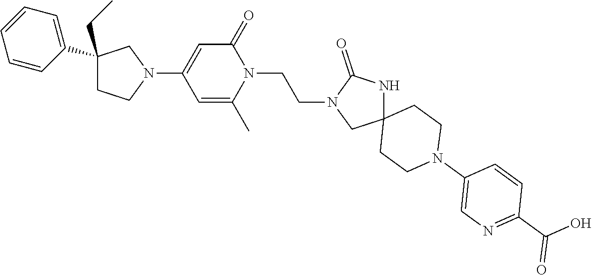 [SMILES: CC[C@]1(c2ccccc2)CCN(c2cc(C)n(CCN3CC4(CCN(c5ccc(C(=O)O)nc5)CC4)NC3=O)c(=O)c2)C1] |
| 20 | 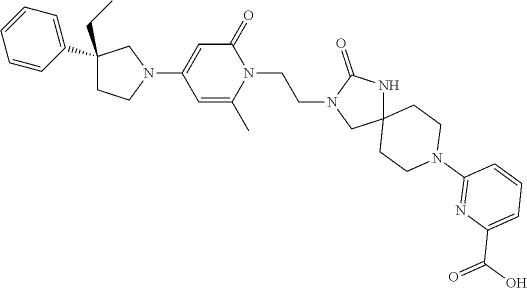 [SMILES: CC[C@]1(c2ccccc2)CCN(c2cc(C)n(CCN3CC4(CCN(c5cccc(C(=O)O)n5)CC4)NC3=O)c(=O)c2)C1] |
| 21 | 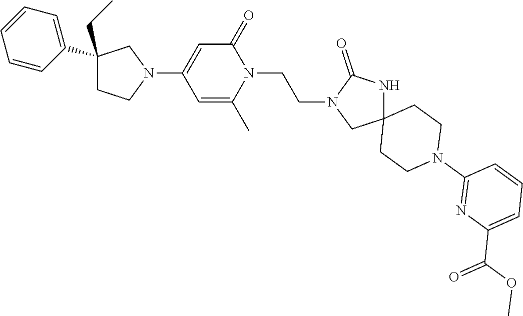 [SMILES: CC[C@]1(c2ccccc2)CCN(c2cc(C)n(CCN3CC4(CCN(c5cccc(C(=O)OC)n5)CC4)NC3=O)c(=O)c2)C1] |
| 22 | 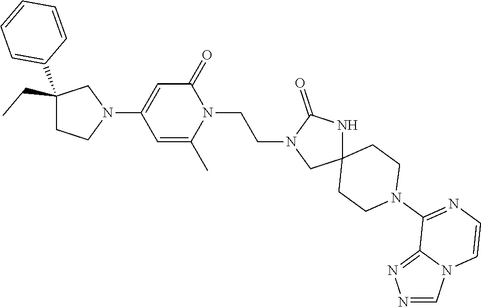 [SMILES: CC[C@]1(c2ccccc2)CCN(c2cc(C)n(CCN3CC4(CCN(c5nccn6cnnc56)CC4)NC3=O)c(=O)c2)C1] |
| 23 | 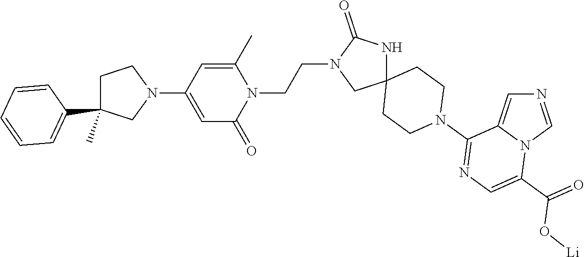 |
| 24 | 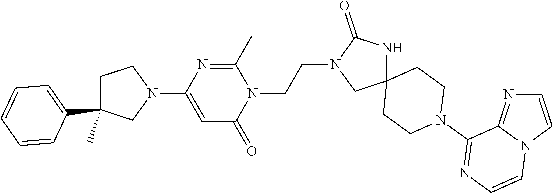 [SMILES: Cc1nc(N2CC[C@](C)(c3ccccc3)C2)cc(=O)n1CCN1CC2(CCN(c3nccn4ccnc34)CC2)NC1=O] |

65. A compound of Formula II: 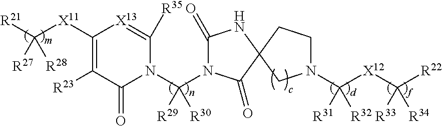
or a pharmaceutically acceptable derivative thereof, wherein:
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
X¹³ is N or CR²⁴;
R²¹ is aryl, heteroaryl or heterocycloalkyl;
R²² is aryl or heteroaryl; and
R²³ to R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

66. The compound of claim 65 that has Formula IIa: 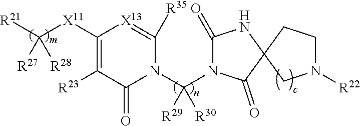
or a pharmaceutically acceptable derivative thereof, wherein:
b is an integer from 1-4;
c is an integer from 1-3;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl;
R²³ to R²⁵, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

67. The compound of claim 65 that has Formula IIb: 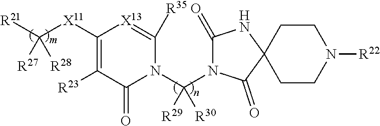
or a pharmaceutically acceptable derivative thereof, wherein:
b is an integer from 1-4;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl; and
R²³ to R²⁵, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

68. The compound of claim 65 that has Formula IIc: 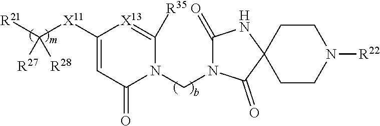
or a pharmaceutically acceptable derivative thereof, wherein:
b is an integer from 1-4;
a is an integer from 0-3;
X¹¹ is a bond, O, NR²⁵ or CO;
X¹³ is N or CH;
R²¹ is aryl, heteroaryl or heterocycloalkyl;
R²² is heteroaryl; and
R²⁵ and R³⁵ are each independently H or alkyl; and
R²⁷ and R²⁸ are each independently H, alkyl or aralkyl, or together with the carbon atom to which they are attached form cycloalkyl.

69. The compound of claim 65, wherein R²¹ is aryl or heterocycloalkyl.

70. The compound of claim 65, wherein R²¹ is aryl.

71. The compound of claim 65, wherein R²¹ is heterocycloalkyl.

72. The compound of claim 65 that has Formula IId: 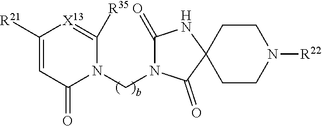
or a pharmaceutically acceptable derivative thereof, wherein:
b is 2 or 3;
X¹³ is N or CH;
R²¹ is heterocycloalkyl, optionally substituted with 1-3 substituents each independently selected from alkyl and aryl;
R²² is heteroaryl; and
R³⁵ is alkyl.

73. The compound of claim 65 that has Formula IIe: 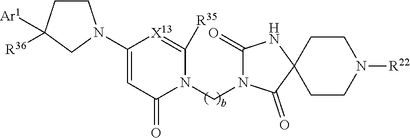
or a pharmaceutically acceptable derivative thereof, wherein:
b is 2 or 3;
X¹³ is N or CH;
Ar¹ is aryl;
R²² is heteroaryl;
R³⁵ is alkyl; and
R³⁶ is alkyl.

74. The compound of claim 73, wherein R³⁶ is lower alkyl.

75. The compound of claim 73, wherein R³⁶ is methyl or ethyl.

76. The compound of claim 73, wherein R³⁶ is methyl.

77. The compound of claim 73, wherein R³⁶ is ethyl.

78. The compound of claim 65 that has the formula:

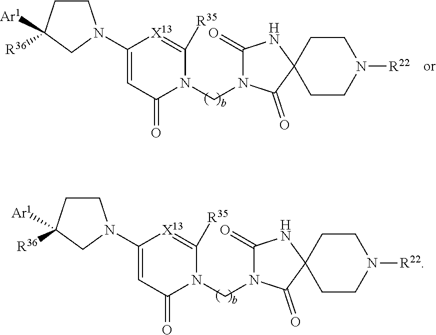

79. The compound of claim 65 that has Formula IIf: 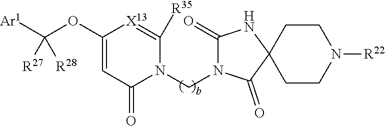
or a pharmaceutically acceptable derivative thereof, wherein:
b is 2 or 3;
X¹³ is N or CH;
Ar¹ is aryl;
R²² is heteroaryl;
R³⁵ is alkyl; and
R²⁷ and R²⁸ are each independently H, alkyl or aralkyl, or together with the carbon atom to which they are attached form cycloalkyl.

80. The compound of claim 65, wherein R²⁷ is alkyl and R²⁸ is H.

81. The compound of claim 65, wherein R²⁷ is methyl and R²⁸ is H.

82. The compound of claim 65, wherein R²⁷ and R²⁸ together with the carbon atom to which they are attached form cyclopropyl.

83. The compound of claim 65 that has the formula:

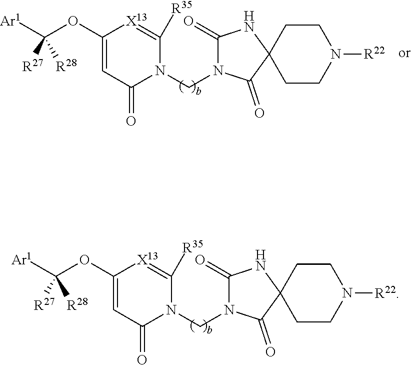

84. The compound of claim 65 that has Formula IIg: 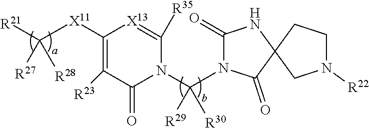
or a pharmaceutically acceptable derivative thereof, wherein:
b is an integer from 1-4;
a is an integer from 0-3;
X¹¹ is a bond, O, NR²⁵ or CO;
X¹³ is N or CR²⁴;
R²¹ is aryl, heteroaryl or heterocycloalkyl;
R²² is heteroaryl;
R²³ to R²⁵, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

85. The compound of claim 65 that has Formula IIh: 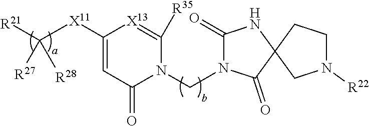
or a pharmaceutically acceptable derivative thereof, wherein:
b is an integer from 1-4;
a is an integer from 0-3;
X¹¹ is a bond, O, NR²⁵ or CO;
X¹³ is N or CH;
R²¹ is aryl, heteroaryl or heterocycloalkyl;
R²² is heteroaryl;
R²⁵ and R³⁵ are each independently H or alkyl; and
R²⁷ and R²⁸ are each independently H, alkyl or aralkyl, or together with the carbon atom to which they are attached form cycloalkyl.

86. The compound of claim 84, wherein R²¹ is aryl or heterocycloalkyl.

87. The compound of claim 84, wherein R²¹ is aryl.

88. The compound of claim 84, wherein R²¹ is heterocycloalkyl.

89. The compound of claim 65 that has Formula IIi: 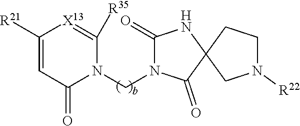
or a pharmaceutically acceptable derivative thereof, wherein:
b is 2 or 3;
X¹³ is N or CH;
R²¹ is heterocycloalkyl, optionally substituted with 1-3 substituents each independently selected from alkyl and aryl;
R²² is heteroaryl; and
R³⁵ is alkyl.

90. The compound of claim 65 that has Formula IIj: 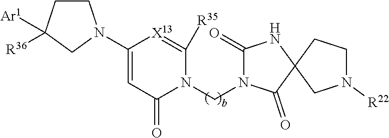
or a pharmaceutically acceptable derivative thereof, wherein:
b is 2 or 3;
X¹³ is N or CH;
Ar¹ is aryl;
R²² is heteroaryl;
R³⁵ is alkyl; and
R³⁶ is alkyl.

91. The compound of claim 90, wherein R³⁶ is lower alkyl.

92. The compound of claim 90, wherein R³⁶ is methyl or ethyl.

93. The compound of claim 90, wherein R³⁶ is methyl.

94. The compound of claim 90, wherein R³⁶ is ethyl.

95. The compound of claim 90 that has the formula:

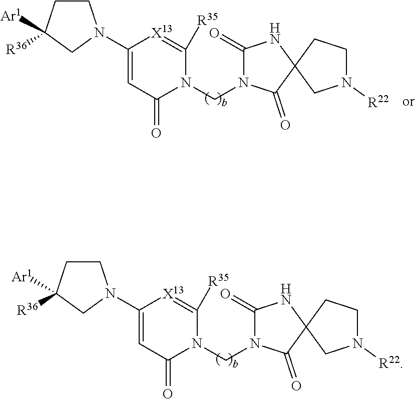

96. The compound of claim 65 that has Formula Ilk: 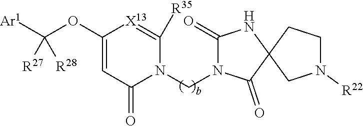
or a pharmaceutically acceptable derivative thereof, wherein:
b is 2 or 3;
X¹³ is N or CH;
Ar¹ is aryl;
R²² is heteroaryl;
R³⁵ is alkyl; and
R²⁷ and R²⁸ are each independently H, alkyl or aralkyl, or together with the carbon atom to which they are attached form cycloalkyl.

97. The compound of claim 65, wherein R²⁷ is alkyl and R²⁸ is H.

98. The compound of claim 65, wherein R²⁷ is methyl and R²⁸ is H.

99. The compound of claim 65, wherein R²⁷ and R²⁸ together with the carbon atom to which they are attached form cyclopropyl.

100. The compound of claim 65 that has the formula:

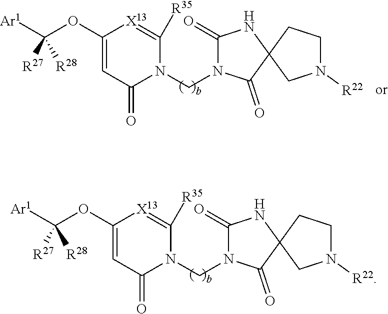

101. The compound of claim 65, wherein b is 2.

102. The compound of claim 65, wherein b is 3.

103. The compound of claim 65, wherein X¹³ is N.

104. The compound of claim 65, wherein X¹³ is CH.

105. The compound of claim 65, wherein Ar¹ is unsubstituted aryl.

106. The compound of claim 65, wherein Ar¹ is optionally substituted phenyl.

107. The compound of claim 65, wherein Ar¹ is unsubstituted phenyl.

108. The compound of claim 65, wherein R²² is bicyclic or monocyclic heteroaryl.

109. The compound of claim 65, wherein R²² is triazolopyrazinyl, imidazopyrazinyl, pyrazolopyrimidinyl, pyridyl, pyridazinyl or pyrimidinyl.

110. The compound of claim 65, wherein R²² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl, 2-pyridyl, 3-pyridyl, 3-pyridazinyl or 2-pyrimidinyl.

111. The compound of claim 65, wherein R²² is [1,2,4]-triazolo[4,3-a]pyrazin-8-yl, imidazo[1,5-a]pyrazin-8-yl, 5-carboxyimidazo[1,5-a]pyrazin-8-yl, imidazo[1,2-a]pyrazin-8-yl, 1H-pyrazolo[3,4-d]pyrimidin-4-yl, 6-carboxy-3-pyridyl, 6-carboxy-2-pyridyl, 6-ethoxycarbonyl-2-pyridyl, 6-ethoxycarbonyl-3-pyridyl, 6-carboxy-3-pyridazinyl, 6-ethoxycarbonyl-3-pyridazinyl or 2-pyrimidinyl.

112. The compound of claim 65, wherein R²² is triazolopyrazinyl, imidazopyrazinyl, pyrazolopyrimidinyl or pyrimidinyl.

113. The compound of claim 65, wherein R²² is [1,2,4]-triazolo[4,3-a]pyrazin-8-yl, imidazo[1,5-a]pyrazin-8-yl, imidazo[1,2-a]pyrazin-8-yl, 1H-pyrazolo[3,4-d]pyrimidin-4-yl or 2-pyrimidinyl.

114. The compound of claim 65, wherein R³⁵ is lower alkyl.

115. The compound of claim 65, wherein R³⁵ is methyl.

116. The compound of claim 65 that has Formula IIm: 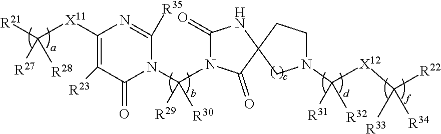
or a pharmaceutically acceptable derivative thereof, wherein
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
R²¹ is aryl, heteroaryl or heterocycloalkyl;
R²² is aryl or heteroaryl;
R²³, R²⁵, R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

117. The compound of claim 65, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

118. The compound of claim 65, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R² is heteroaryl;
R²³ and R²⁸ to R³⁰ are each independently H; and
R²⁷ and R³⁵ are each independently alkyl.

119. The compound of claim 65, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁹, R³⁰ and R³⁵ are each independently H, alkyl or aralkyl; and
R²⁷ and R²⁸ are each independently H, alkyl or aralkyl, or together with the carbon atom to which they are attached form cycloalkyl.

120. The compound of claim 65, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R² is heteroaryl;
R²³ and R²⁸ to R³⁰ are each independently H; and
R²⁷ and R³⁵ are each independently alkyl.

121. The compound of claim 65 that has Formula IIn: 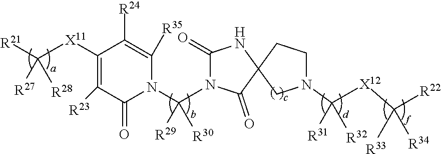
or a pharmaceutically acceptable derivative thereof, wherein
b is an integer from 1-4;
c is an integer from 1-3;
a, d and f are each independently an integer from 0-3;
X¹¹ is a bond, O, NR²⁵ or CO;
X¹² is a bond, CONR²⁶, SO₂NR²⁶, CO or SO₂;
R²¹ is aryl, heteroaryl or heterocycloalkyl;
R²² is aryl or heteroaryl; and
R²³ to R²⁶ and R²⁹ to R³⁵ are each independently H, alkyl or aralkyl; and
R²⁷ and R²⁸ are each independently H, alkyl or aralkyl, or together with the carbon atom to which they are attached form cycloalkyl.

122. The compound of claim 65, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴, R²⁹, R³⁰, and R³⁵ are each independently H, alkyl or aralkyl; and
R²⁷ and R²⁸ are each independently H, alkyl or aralkyl, or together with the carbon atom to which they are attached form cycloalkyl.

123. The compound of claim 65, or a pharmaceutically acceptable derivative thereof, wherein:
b is 2 or 3;
c is 2;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴ and R²⁸ to R³⁰ are each independently H; and
R²⁷ and R³⁵ are each independently alkyl.

124. The compound of claim 65, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴, R²⁹, R³⁰, and R³⁵ are each independently H, alkyl or aralkyl; and
R²⁷ and R²⁸ are each independently H, alkyl or aralkyl, or together with the carbon atom to which they are attached form cycloalkyl.

125. The compound of claim 65, or a pharmaceutically acceptable derivative thereof, wherein:
b is 2 or 3;
c is 1;
a is 0 or 1;
d and f are each 0;
X¹¹ is a bond or O;
X¹² is a bond;
R²¹ is aryl or heterocycloalkyl, each optionally substituted with 1-3 substituents each independently selected from alkyl and phenyl;
R²² is heteroaryl; and
R²³, R²⁴ and R²⁸ to R³⁰ are each independently H; and
R²⁷ and R³⁵ are each independently alkyl.

126. The compound of claim 65 that has Formula IIo: 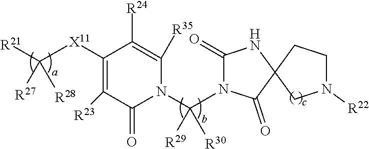
or a pharmaceutically acceptable derivative thereof, wherein:
b is an integer from 1-4;
c is an integer from 1-3;
a is an integer from 0-3;
X¹¹ is a bond, O, NR²⁵ or CO;
R²¹ is aryl or heterocycloalkyl;
R²² is heteroaryl; and
R²³ to R²⁵, R²⁷ to R³⁰ and R³⁵ are each independently H or alkyl.

127. The compound of claim 65 that has Formula IIp: 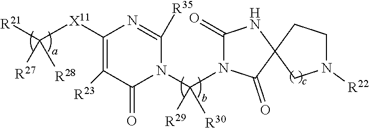
or a pharmaceutically acceptable derivative thereof, wherein:
b is an integer from 1-4;
c is an integer from 1-3;
a is an integer from 0-3;
X¹¹ is a bond, O, NR²⁵ or CO;
R²¹ is aryl or heterocycloalkyl;
R²² is heteroaryl; and
R²³, R²⁵, R²⁷ to R³⁰ and R³⁵ are each independently H or alkyl.

128. The compound of claim 126, wherein:
b is 2 or 3;
c is 2;
a is 0 or 1;
X¹¹ is a bond or O;
R²¹ is aryl or heterocycloalkyl, each optionally substituted with 1-3 substituents each independently selected from alkyl and aryl;
R²² is heteroaryl; and
R²³, R²⁵, R²⁷ to R³⁰ and R³⁵ are each independently H or alkyl.

129. The compound of claim 126, wherein:
b is 2 or 3;
c is 1;
a is 0 or 1;
X¹¹ is a bond or O;
R²¹ is aryl or heterocycloalkyl, each optionally substituted with 1-3 substituents each independently selected from alkyl and aryl;
R²² is heteroaryl; and
R²³, R²⁵, R²⁷ to R³⁰ and R³⁵ are each independently H or alkyl.

130. The compound of claim 65 that is:

| Compound | Structure |
|---|---|
| 25 | 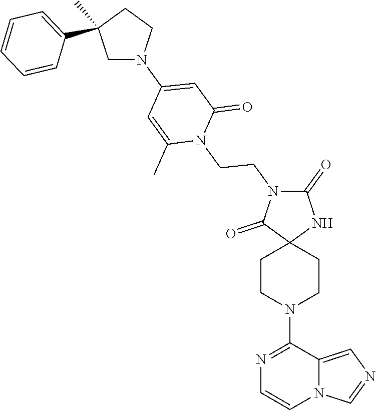 [SMILES: Cc1cc(N2CC[C@](C)(c3ccccc3)C2)cc(=O)n1CCN1C(=O)NC2(CCN(c3nccn4cncc34)CC2)C1=O] |
| 26 | 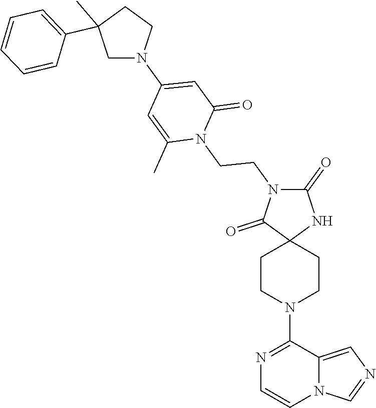 [SMILES: Cc1cc(N2CCC(C)(c3ccccc3)C2)cc(=O)n1CCN1C(=O)NC2(CCN(c3nccn4cncc34)CC2)C1=O] |
| 27 | 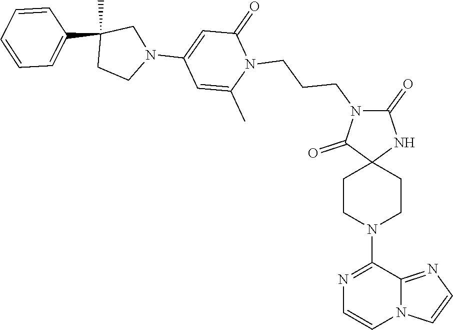 [SMILES: Cc1cc(N2CC[C@@](C)(c3ccccc3)C2)cc(=O)n1CCCN1C(=O)NC2(CCN(c3nccn4ccnc34)CC2)C1=O] |
| 28 | 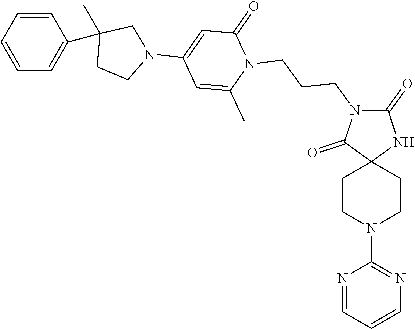 [SMILES: Cc1cc(N2CCC(C)(c3ccccc3)C2)cc(=O)n1CCCN1C(=O)NC2(CCN(c3ncccn3)CC2)C1=O] |
| 29 | 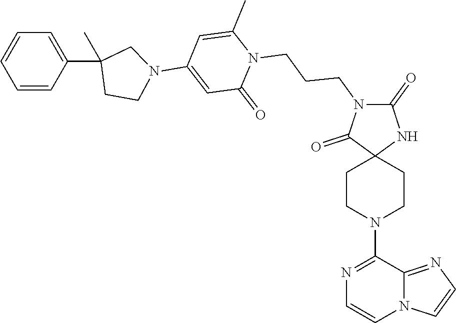 [SMILES: Cc1cc(N2CCC(C)(c3ccccc3)C2)cc(=O)n1CCCN1C(=O)NC2(CCN(c3nccn4ccnc34)CC2)C1=O] |
| 30 | 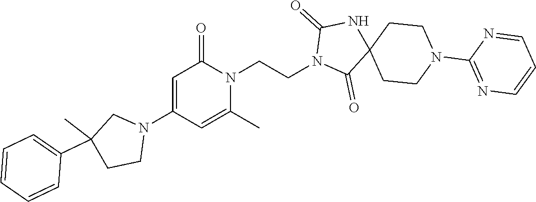 [SMILES: Cc1cc(N2CCC(C)(c3ccccc3)C2)cc(=O)n1CCN1C(=O)NC2(CCN(c3ncccn3)CC2)C1=O] |
| 31 | 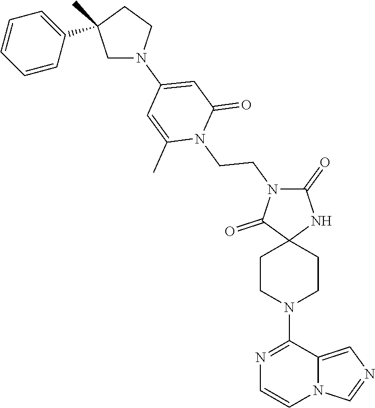 [SMILES: Cc1cc(N2CC[C@@](C)(c3ccccc3)C2)cc(=O)n1CCN1C(=O)NC2(CCN(c3nccn4cncc34)CC2)C1=O] |
| 32 | 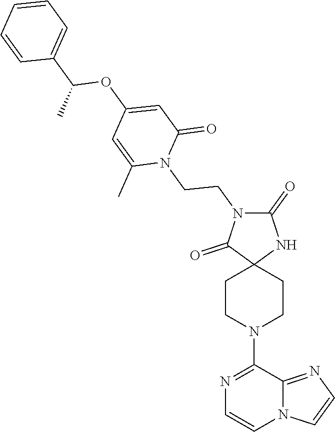 [SMILES: Cc1cc(O[C@H](C)c2ccccc2)cc(=O)n1CCN1C(=O)NC2(CCN(c3nccn4ccnc34)CC2)C1=O] |
| 33 | 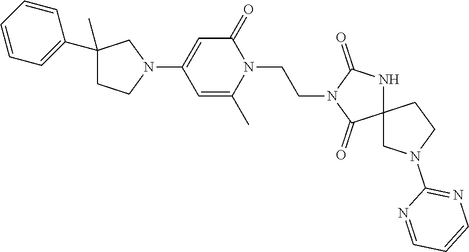 [SMILES: Cc1cc(N2CCC(C)(c3ccccc3)C2)cc(=O)n1CCN1C(=O)NC2(CCN(c3ncccn3)C2)C1=O] |
| 34 | 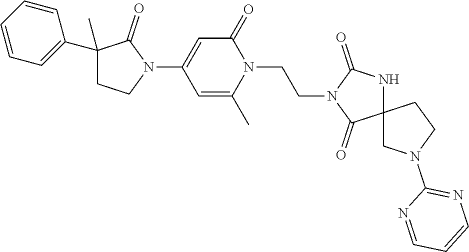 [SMILES: Cc1cc(N2CCC(C)(c3ccccc3)C2=O)cc(=O)n1CCN1C(=O)NC2(CCN(c3ncccn3)C2)C1=O] |
| 35 | 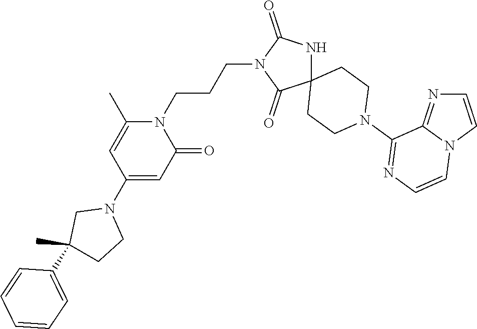 [SMILES: Cc1cc(N2CC[C@](C)(c3ccccc3)C2)cc(=O)n1CCCN1C(=O)NC2(CCN(c3nccn4ccnc34)CC2)C1=O] |

131. A pharmaceutical composition, comprising the compound of claim 1 and a pharmaceutically acceptable carrier.

132. A method of treating light chain amyloidosis, comprising administering to a subject a compound of claim 1.

133. A method of stabilizing immunoglobulin light chains, comprising contacting the immunoglobulin light chains with a compound of claim 1.

134. The method of claim 133, wherein the immunoglobulin light chains are stabilized in a native conformation thereof.

135. The method of claim 133, wherein the immunoglobulin light chains are dimers.

136. A method of preventing or lessening immunoglobulin light chain misfolding and/or endoproteolysis, comprising contacting the immunoglobulin light chains with a compound of claim 1.

137. A method of maintenance therapy upon recurrence of light chain amyloidosis following primary treatment, comprising administering to a subject a compound of a claim 1.

138. The method of claim 132, further comprising administering to the subject a second active agent selected from proteasome inhibitors (e.g., bortezomib, ixazomib, carfilzomib), alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129), an agent that promotes amyloid deposit clearance (e.g., CAEL-101, birtamimab), an anti-thymocyte antibody (e.g., Thymoglobulin®, Atgam®), atacicept and/or an anti-amyloid antibody.

139. The method of claim 132, further comprising stem cell transplant therapy.

140. The method of claim 138, wherein the second active agent is a plasma cell-directed therapy.

141. A pharmaceutical composition, comprising the compound of claim 65 and a pharmaceutically acceptable carrier.

142. A method of treating light chain amyloidosis, comprising administering to a subject a compound of claim 65.

143. A method of stabilizing immunoglobulin light chains, comprising contacting the immunoglobulin light chains with a compound of claim 65.

144. The method of claim 143, wherein the immunoglobulin light chains are stabilized in a native conformation thereof.

145. The method of claim 143, wherein the immunoglobulin light chains are dimers.

146. A method of preventing or lessening immunoglobulin light chain misfolding and/or endoproteolysis, comprising contacting the immunoglobulin light chains with a compound of claim 65.

147. A method of maintenance therapy upon recurrence of light chain amyloidosis following primary treatment, comprising administering to a subject a compound of a claim 65.

148. The method of claim 142, further comprising administering to the subject a second active agent selected from proteasome inhibitors (e.g., bortezomib, ixazomib, carfilzomib), alkylating agents (e.g., bendamustine, melphalan, cyclophosphamide), steroids (e.g., dexamethasone), immunomodulatory agents (e.g., thalidomide, lenalidomide, pomalidomide), an anti-CD38 antibody (e.g., daratumumab, isatuximab), an anti-CD20 antibody (e.g., rituximab), an anti-IL-6 antibody (e.g., siltuximab), a UPR activator (e.g., an ATF-6 activator), an antibody-drug-conjugate (e.g., belantamab mafodotin, STI-6129), an agent that promotes amyloid deposit clearance (e.g., CAEL-101, birtamimab), an anti-thymocyte antibody (e.g., Thymoglobulin®, Atgam®), atacicept and/or an anti-amyloid antibody.

149. The method of claim 142, further comprising stem cell transplant therapy.

150. The method of claim 148, wherein the second active agent is a plasma cell-directed therapy.
